SEQUENCE LISTING (23 sequences)

SEQ ID NO: 1 - protein, 87 aa, Artificial
  partial region of fibronectin named III-8
PTDLRFTNIGPDTMRVTWAPPPSIDLTNFLVRYSPVKNEEDVAELSISPSDNAVVLTNLLPGTEYVVSVSSVYEQHESTPLRGRQKT

SEQ ID NO: 2 - protein, 90 aa, Artificial
  partial region of fibronectin named III-9
GLDSPTGIDFSDITANSFTVHWIAPRATITGYRIRHHPEHFSGRPREDRVPHSRNSITLTNLTPGTEYVVSIVALNGREESPLLIGQQST

SEQ ID NO: 3 - protein, 94 aa, Artificial
  partial region of fibronectin named III-10
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 4 - protein, 84 aa, Artificial
  partial region of fibronectin named III-11
QMQVTDVQDNSISVKWLPSSSPVTGYRVTTTPKNGPGPTKTKTAGPDQTEMTIEGLQPTVEYVVSVYAQNPSGESQPLVQTAVT

SEQ ID NO: 5 - protein, 92 aa, Artificial
  partial region of fibronectin named III-12
AIPAPTDLKFTQVTPTSLSAQWTPPNVQLTGYRVRVTPKEKTGPMKEINLAPDSSSVVVSGLMVATKYEVSVYALKDTLTSRPAQGVVTTLE

SEQ ID NO: 6 - protein, 89 aa, Artificial
  partial region of fibronectin named III-13
NVSPPRRARVTDATETTITISWRTKTETITGFQVDAVPANGQTPIQRTIKPDVRSYTITGLQPGTDYKIYLYTLNDNARSSPVVIDAST

SEQ ID NO: 7 - protein, 90 aa, Artificial
  partial region of fibronectin named III-14
AIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 8 - protein, 25 aa, Artificial
  partial region of fibronectin named CS-1
DELPQLVTLPHPNLHGPEILDVPST

SEQ ID NO: 9 - protein, 274 aa, Artificial
  fibronectin fragment named C-274
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEID

SEQ ID NO: 10 - protein, 271 aa, Artificial
  fibronectin fragment named H-271
AIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 11 - protein, 296 aa, Artificial
  fibronectin fragment named H-296
AIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPST

SEQ ID NO: 12 - protein, 549 aa, Artificial
  fibronectin fragment named CH-271
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 13 - protein, 574 aa, Artificial
  fibronectin fragment named CH-296
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPST

SEQ ID NO: 14 - protein, 302 aa, Artificial
  fibronectin fragment named C-CS1
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSDELPQLVTLPHPNLHGPEILDVPST

SEQ ID NO: 15 - protein, 658 aa, Artificial
  fibronectin fragment named CH-296Na
MPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPST

SEQ ID NO: 16 - protein, 367 aa, Artificial
  fibronectin fragment named CHV-89
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMNVSPPRRARVTDATETTITISWRTKTETITGFQVDAVPANGQTPIQRTIKPDVRSYTITGLQPGTDYKIYLYTLNDNARSSPVVIDAST

SEQ ID NO: 17 - protein, 368 aa, Artificial
  fibronectin fragment named CHV-90
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMAIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 18 - protein, 370 aa, Artificial
  fibronectin fragment named CHV-92
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMAIPAPTDLKFTQVTPTSLSAQWTPPNVQLTGYRVRVTPKEKTGPMKEINLAPDSSSVVVSGLMVATKYEVSVYALKDTLTSRPAQGVVTTLE

SEQ ID NO: 19 - protein, 457 aa, Artificial
  fibronectin fragment named CHV-179
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMNVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 20 - protein, 459 aa, Artificial
  fibronectin fragment named CHV-181
PTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSMAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDAST

SEQ ID NO: 21 - protein, 276 aa, Artificial
  fibronectin fragment named H-275-Cys
MAASAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTC

SEQ ID NO: 22 - protein, 594 aa, Artificial
  fibronectin fragment named H296-H296
AIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTAMAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPST

SEQ ID NO: 23 - protein, 234 aa, Artificial
  fibronectin fragment named H105-H105
HMAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTHMAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPST

(19) United States
(12) Patent Application Publication
(10) Pub. No.: US 2010/0247579 A1
(43) Pub. Date: Sep. 30, 2010

(54) THERAPEUTIC AGENT FOR CANCER

(76) Inventors: Hiroshi Shiku, Mie (JP); Shinichi Kageyama, Mie (JP); Shigehisa Kitano, Mie (JP); Mitsuko Ideno, Shiga (JP); Keisuke Tomita, Shiga (JP); Tatsuji Enoki, Shiga (JP); Kazutoh Takesako, Shiga (JP); Ikunoshin Kato, Shiga (JP)

Correspondence Address: BROWDY AND NEIMARK, P.L.L.C.;624 NINTH STREET, NW, SUITE 300, WASHINGTON, DC, 20001-5303, US

(21) Appl. No.: 12/599,541

(22) PCT Filed: May 8, 2008
(86) PCT No.: PCT/JP2008/058568

§ 371 Date: May 25, 2010

(30) Foreign Application Priority Data
May 11, 2007 (JP) 2007-127436

(51) Int. Cl. A61K 39/00; A61K 35/12; C12N 5/00; A61P 35/04

(52) U.S. Cl. 424/277.1; 424/93.71; 435/325

(57) ABSTRACT

The present invention relates to a method for treatment of cancer, which comprises the following steps (A) and (B): (A) applying a treatment which induces the reduction in lymphocytes to a patient; and (B), subsequent to step (A), administering lymphocytes to the patient promptly. The present invention also relates to a cancer therapeutic agent and a cancer treatment kit for use in the method. When the method is used as an immunoreconstructive therapy, the decrease in immunologic competence which may be caused by the reduction in lymphocytes can be avoided and, therefore, the risk of the occurrence of an infectious disease can be decreased.

### TECHNICAL FIELD

The present invention relates to a cancer therapeutic agent and a treatment method, which are useful in the medical field.

### BACKGROUND ART

An operative therapy, a radiotherapy, a chemotherapy, an immunotherapy (a cell therapy or a vaccine therapy), etc. have been used for the cancer therapy. Among them, a chemotherapy with an anticancer agent is common. Many of anticancer agents have a lot of side effects because they damage not only cancer cells but also normal cells actively proliferating. For example, a side effect that is called a hematologic toxicity due to bone marrow suppression can be caused, and, as a result, neutrophils, platelets, lymphocytes, etc. in the peripheral blood are decreased below the normal value. In such a case, it is effective to repeat administration with a certain convalescent period so that anticancer agents in as large amount as possible may be administered, while suppressing the side effect to the minimum, and thus this method has been often employed. Moreover, in the chemotherapy using an anticancer agent, two or more anticancer agents are usually used in combination.

In an adoptive immunotherapy that is a kind of the cell therapies which has been often applied to cancer patients, a patient's own lymphocytes are cultured ex vivo, and the obtained lymphocytes are administered to the patient. As the culture method, there are various methods, and addition of lymphocyte growth factors including interleukin-2 (IL-2) and interleukin-15 (IL-15), and stimulation with an anti-CD3 antibody or co-stimulation of an anti-CD3 antibody and an anti-CD28 antibody in combination with a lymphocyte growth factor, and the like, are mainly used. In addition, culture has also been performed with adding, as an antigen, a tumor cell, a tumor antigen protein or peptide, or an antigen presenting cell treated with an antigen so that the lymphocytes may be educated to recognize or damage a tumor.

Among the adoptive immunotherapies, in the therapy of transferring a lymphokine-activated cell obtained from cytotoxic T lymphocytes (CTL) or peripheral blood lymphocytes which have been induced ex vivo, by expansion through the action of IL-2 and anti-CD3 antibody, the present inventors have already studied the effect of use of fibronectin and its fragment on the problems about how to maintain the cytotoxicity in the expansion of the cells, how to enable to make the lymphocytes expanded ex vivo efficiently, and how to establish efficiently the expansion of the lymphocytes having an ability suitable for therapy, and the like (for example, see Patent Documents 1 to 6).

In general, a combination of the adoptive immunotherapy with an anticancer agent having a strong hematologic toxicity is not used so that the transferred cells may not be killed by the cellular cytotoxicity and hematologic toxicity, etc. of the anticancer agent used. The adoptive immunotherapy to a cancer patient to whom an anticancer agent has been administered is performed at a sufficient interval after use of the anticancer agent. That is, the adoptive immunotherapy is carried out after the completion of the treatment period with anticancer agents.

In the vaccine therapy, a tumor antigen protein or an antigen peptide derived therefrom is formulated in admixture with an adjuvant (incomplete Freund's adjuvant, CpG, etc.) to improve the immunogenicity, and such a formulation is then used. Other than these, a derivative (a chimera protein with GM-CSF, etc.) for enhancing antigen immunogenicity, antigen presenting cells incorporated with a protein or a peptide, and a DNA vaccine for expressing an antigen gene have been examined, and these have been used alone or in admixture with an adjuvant. These antigens used in the above vaccine have been all prepared ex vivo and administered.

Patent Document 1: WO 03/016511

Patent Document 2: WO 03/080817

Patent Document 3: WO 2005/019450

Patent Document 4: Japanese Patent Publication No. 2007-061020

Patent Document 5: WO 2007/020880

Patent Document 6: WO 2007/040105

### DISCLOSURE OF THE INVENTION

### Problems to be Solved by the Invention

Although anticancer agents show the effect by killing cancer cells, many of the anticancer agents kill normal cells as well as cancer cells. On this occasion, blood cells such as lymphocytes are killed, and the leukocyte count is decreased. Therefore, the anticancer agent is administered in several cycles with a certain convalescent period after the recovery of the cell function affected by the anticancer agent, with the assumption of the metabolism of the anticancer agent in the body of a patient, but there are some cases where a sufficient killing effect for cancer cells cannot be achieved.

On the other hand, treatment with an anticancer agent causes a reduction in the immune function due to a decrease of leukocytes including the lymphocytes, and increases the risk of infectious diseases. Moreover, such treatment may cause the delayed recovery of immunoreaction to the cancer cell.

An object of the present invention is to provide a method of treating cancer, a therapeutic agent for cancer, and a cancer treatment kit, which are effective for administration to the living body.

### Means of Solving the Problems

A first invention according to the present invention relates to a method of treating cancer, comprising the following steps (A) and (B):

(A) applying a treatment which induces the reduction in lymphocytes to a patient, and

(B) subsequent to the step (A), administering lymphocytes to the patient promptly. In the first invention according to the present invention, examples of the treatment which induces the reduction in lymphocytes include administration of an anticancer agent and/or radiation. In addition, examples of the anticancer agent include an anticancer agent selected from a group consisting of anticancer agents classified into a metabolic antagonist, an antibiotic (an antitumor antibiotic), a microtubule inhibitor, a topoisomerase inhibitor, a platinum preparation, an alkylating agent or a corticosteroid, and in a preferred aspect, examples of the anticancer agent include at least one anticancer agent selected from a group consisting of fluorouracil, methotrexate, gemcitabine, fludarabine, bleomycin, adriamycin, mitomycin, paclitaxel, docetaxel, vincristine, irinotecan, etoposide, cisplatin, carboplatin, nedaplatin, doxorubicin, dexamethasone and cyclophosphamide. Moreover, in the first invention according to the present invention, an aspect that the step (B) is carried out one hour to 10 days after the step (A) is exemplified. Moreover, in the first invention according to the present invention, examples of the lymphocyte to be administered include a lymphocyte-containing culture, and in particular, a lymphocyte culture obtained by culturing lymphocytes in the presence of an anti-CD3 antibody. In addition, as the lymphocytes to be administered, there is exemplified a lymphocyte culture obtained by culturing lymphocytes collected from a patient. Moreover, a lymphocyte culture obtained by culturing lymphocytes in the presence of fibronectin, a fibronectin fragment, or a mixture thereof is exemplified as the lymphocytes to be administered. In the first invention according to the present invention, an aspect further comprising the step of administering a cancer vaccine and/or a lymphocyte stimulating factor during or after the step (B) is exemplified.

A second invention according to the present invention relates to a lymphocyte-containing therapeutic agent for cancer which can be administered promptly to a patient to whom a treatment inducing the reduction in lymphocytes has been applied, subsequent to the treatment. In the second invention according to the present invention, examples of the treatment inducing the reduction in lymphocytes include administration of an anticancer agent and/or radiation. In addition, in the second invention according to the present invention, exemplified is a lymphocyte-containing therapeutic agent for cancer which is administered to a patient to whom a treatment inducing the reduction in lymphocytes has been applied one hour to 10 days after such a treatment. Moreover, as the lymphocytes, a culture is exemplified and in particular, a lymphocyte culture obtained by culturing lymphocytes in the presence of an anti-CD3 antibody is exemplified. Also, as the lymphocyte, there is exemplified a lymphocyte culture obtained by culturing lymphocytes collected from a patient. In addition, examples of the lymphocytes include a lymphocyte culture obtained by culturing lymphocytes in the presence of fibronectin, a fibronectin fragment, or a mixture thereof.

A third invention according to the present invention relates to a cancer treatment kit, including separately an anticancer agent which causes the reduction in lymphocytes and the therapeutic agent of the second invention according to the present invention. In the third invention according to the present invention, examples of the anticancer agent include at least one anticancer agent selected from a group consisting of anticancer agents classified into a metabolic antagonist, an antibiotic (an antitumor antibiotic), a microtubule inhibitor, a topoisomerase inhibitor, a platinum preparation, an alkylating agent or a corticosteroid, and in a preferred aspect, examples of the anticancer agent include at least one anticancer agent selected from a group consisting of fluorouracil, methotrexate, gemcitabine, fludarabine, bleomycin, adriamycin, mitomycin, paclitaxel, docetaxel, vincristine, irinotecan, etoposide, cisplatin, carboplatin, nedaplatin, doxorubicin, dexamethasone and cyclophosphamide.

A fourth invention according to the present invention relates to a cancer treatment kit, including separately the therapeutic agent of the second invention according to the present invention, and a cancer vaccine and/or a lymphocyte stimulating factor.

A fifth invention according to the present invention relates to a cancer treatment kit, including separately the cancer treatment kit of the third invention according to the present invention, and a cancer vaccine and/or a lymphocyte stimulating factor.

A sixth invention according to the present invention relates to use of lymphocytes in the production of the therapeutic agent of the second invention according to the present invention.

A seventh invention according to the present invention relates to use of an anticancer agent which causes the reduction in lymphocytes, and lymphocytes in the production of the cancer treatment kit according to the third invention of the present invention.

An eighth invention according to the present invention relates to use of lymphocytes, and a cancer vaccine and/or a lymphocyte stimulating factor in the production of the cancer treatment kit according to the fourth invention of the present invention.

A ninth invention according to the present invention relates to use of an anticancer agent which causes the reduction in lymphocytes, lymphocytes, and a cancer vaccine and/or a lymphocyte stimulating factor in the production of the cancer treatment kit according to the fifth invention of the present invention.

### EFFECT OF THE INVENTION

According to the present invention, a method of treating cancer and a cancer therapeutic agent, which activate cellular immunity against cancer and have a high therapeutic effect is provided. A treatment method for imparting damage to cancer cells including an anticancer agent damages the cancer cells to release a large amount of tumor antigens into the body. The lymphocytes which are administered under these situations cause an immunoresponse to the released tumor antigen, thereby acquiring the cytotoxicity against the cancer cells. In other words, the lymphocytes to be administered are a not yet known anticancer agent that functions as a new vaccine therapy. Moreover, the treatment method and the therapeutic agent can reduce the risk of infectious diseases because a decrease in immunity due to the reduction in lymphocytes can be avoided.

### BEST MODE FOR CARRYING OUT THE INVENTION

The present invention provides a method of treating cancer, including the following steps:

(A) applying a treatment which induces the reduction in lymphocytes to a patient; and

(B) subsequent to the step (A), administering lymphocytes to the patient promptly.

There is no particular limitation to the treatment performed in the step (A) as long as it causes a reduction in lymphocytes in a patient as a result of the practice for treating a patient. The above-mentioned treatment is usually applied for suppressing the proliferation of cancer cells or killing the cells, and for example, administration of anticancer agents and radiation are exemplified. Examples of the anticancer agent which causes the reduction in lymphocytes in a patient to whom it is administered include a metabolic antagonist (fluorouracil, methotrexate, gemcitabine, fludarabine), an antibiotic (bleomycin, adriamycin, mitomycin), a microtubule inhibitor (paclitaxel, docetaxel, vincristine), a topoisomerase inhibitor (irinotecan, etoposide, doxorubicin), a platinum preparation (cisplatin, carboplatin, nedaplatin), an alkylating agent (cyclophosphamide), or a corticosteroid (dexamethasone), etc, although the present invention is not particularly limited to these examples. In addition, the present invention also includes an aspect wherein these anticancer agents specifically mentioned herein are used in forms of pharmaceutically acceptable esters and/or pharmaceutically acceptable salts thereof. These anticancer agents may be administered alone or in appropriate combinations, or they may be used in combination with an anticancer agent which causes the reduction in lymphocytes and another anticancer agent which does not cause the reduction in lymphocytes. Moreover, the anticancer agent may include a cancer metastasis inhibitor as long as it causes the reduction in lymphocytes in a patient to whom it has been administered.

In the present invention, the reduction in lymphocytes means the decrease in lymphocyte count in blood compared with that before the practice of the step (A). For instance, it means a state where the blood lymphocyte count is decreased to 1000/μL or less in adults and 3000/μL or less in children.

The step (A) may be a single time treatment or may be repeated multiple times. The frequency and the condition for the treatment, e.g. the doses of the anticancer agent, are determined by taking the action against cancer cells and the damage to the patient into consideration. For example, in the case where an anticancer agent is administered in the step (A), such an anticancer agent is administered in several divided doses, e.g. 2 to 5 divided doses, and then the process is advanced to the step (B). All the doses of the anticancer agents may be all equal, alternatively the second or later dose may be reduced compared to the first dose. Moreover, when the step (A) is administration of an anticancer agent, its administration mode is not particularly limited and a known administration method and dose may be used.

The lymphocyte to be administered to a patient in the step (B) is not particularly limited, as long as it can reconstruct the immune function of the patient affected by the reduction in lymphocytes, caused in the step (A), i.e., as long as it can prevent or alleviate the reduction in the immune function to allow the patient to maintain or recover the immunity. A cell population containing lymphocytes may be used as the above-mentioned lymphocytes. For example, a cell population containing lymphocytes fractionated from materials such as peripheral blood, umbilical cord blood, and bone marrow by a known method, and a cell population containing progenitor cells of lymphocytes derived from the materials, for example, a cell population containing lymphocytes prepared from mononuclear cells are exemplified. In addition, the above materials may be either those collected from the patient (autologous lymphocytes) or those collected from a donor other than the patient (donor lymphocytes), but a material collected from the patient is preferably used. When the above-mentioned material or lymphocyte is collected from the patient, the collection may be carried out either before or after the step (A). In addition, the lymphocyte to be administered to a patient in the step (B) may be a foreign gene-transferred lymphocyte. Further, the “foreign gene” means a gene which is artificially transferred into lymphocytes into which the gene is to be transferred, and also encompasses a gene derived from the same species as the one from which lymphocytes into which the gene is to be transferred is derived.

Moreover, the dose of a lymphocyte administered to a patient in the step (B) and the various conditions may be determined according to the immune status. Although the present invention is not particularly limited, for example, the daily dose of lymphocytes per adult may preferably be 1×10⁵ to 1×10¹² cells/day, more preferably be 1×10⁶ to 5×10¹¹ cells/day, and still more preferably be 1×10⁶ to 1×10¹¹ cells/day. In addition, the dose may vary in accordance with the treatment in the step (A). The lymphocytes are usually administered intravenously, intraarterially, subcutaneously, and intraperitoneally via injection or drop infusion.

Furthermore, the lymphocyte-containing cell population may be a culture obtained by subjecting a suitable cell population to an artificial cell culture procedure. A preferred aspect is exemplified by a method of treating cancer including using the conditions to expand the lymphocytes in the above cultivation and administering the resultant culture to a patient. For example, a cell population obtained by culturing a material containing a lymphocyte or a lymphocyte progenitor (e.g. a peripheral blood mononuclear cell, an umbilical cord blood mononuclear cell, or a hematopoietic stem cell, etc.) in the presence of a known lymphocyte-stimulating factor or cofactor [e.g. an anti-CD3 antibody, an anti-CD28 antibody, a cytokine (IL-2, IL-15, interleukin-7 (IL-7), interleukin-12 (IL-12), interferon-γ (IFN-γ), interferon-α (IFN-α), or interferon-β (IFN-β)), or a chemokine, etc.] can be used in the treatment method of the present invention. A cell population obtained by culturing lymphocytes in the presence of IL-2 and an anti-CD3 antibody is preferably exemplified.

The culture obtained from an artificial cell culture procedure, which is used in the treatment method of the present invention can be, for example, a cell population obtained by culturing lymphocytes in the presence of fibronectin, a fibronectin fragment or a mixture thereof. The fibronectin fragment can be exemplified by a fragment containing the amino acid sequences as shown in SEQ ID NOs: 1 to 8 of Sequence Listings (III-8, III-9, III-10, III-11, III-12, III-13, III-14, CS-1 domains of the fibronectin), and preferable examples of the fibronectin fragment include a fragment containing any one of cell-binding domains (III-8 to III-10 domains) of the fibronectin, heparin-binding domains (III-12 to III-14 domains) or CS-1 domain. The fibronectin fragment may also be a fragment having overlapped amino acid sequences as shown by SEQ ID NOs: 1 to 8 of Sequence Listings. In particular, a preferred fibronectin fragment used in the present invention may be a fragment having the amino acid sequences as shown in SEQ ID NOs: 9 to 23 or a polypeptide containing the amino acid sequences having substitution, deletion, insertion or addition of one or more amino acids in the amino acid sequences of the polypeptide constituting the fragment, wherein the polypeptide has a function equivalent to the above-exemplified fibronectin fragment.

It is preferable that the substitution or the like of the amino acids is carried out to an extent that it can change physicochemical characteristics and the like of a polypeptide as long as the inherent function of the polypeptide can be maintained. For example, it is preferable that the substitution or the like of the amino acids is conservative, within the range that the characteristics inherently owned by the polypeptide (for example, hydrophobicity, hydrophilicity, electric charge, pK, etc.) are not substantially changed. For example, it is preferable that the substitution of the amino acids is substitutions with another amino acid belonging to the same group shown below: group 1. glycine, alanine; 2. valine, isoleucine, leucine; 3. aspartic acid, glutamic acid, asparagine, glutamine; 4. serine, threonine; 5. lysine, arginine; 6. phenylalanine, tyrosine, and that the deletion, addition or insertion of the amino acids is such that the deletion, addition or insertion of the amino acids having characteristics similar to the characteristics of the surroundings of the targeted site in the polypeptide as long as the characteristics of the surroundings of the targeted site are not substantially changed.

The production of a cell population by cultivation in the presence of fibronectin, a fibronectin fragment or a mixture thereof is carried out, for example, according to the methods described in WO 03/016511, WO 03/080817, WO 03/019450, Japanese Patent Publication No. 2007-061020, WO 2007/020880, or WO 2007/040105.

The cell population containing lymphocytes for use in the present invention is preferably a cell population containing T cells at a high rate. Particularly preferred is a cell population containing naive T cells or T cells which express surface antigen markers of naive T cells (hereinafter referred to as naive T-like cells), such as CD45RA , CD62L, CCR7, CD27, CD28, etc., at a high rate. In the body of the patient after the step (A), a high proportion of cancer antigens released from the killed cancer cells are contained in the blood and they are ingested by antigen-presenting cells such as macrophages or dendritic cells, etc., resulting in the state where cancer antigens are presented at a high proportion, which makes it easy to induce CTL having a cancer antigen-specific cytotoxicity. Accordingly, by administering a cell population which contains a naive T cell or a naive T-like cell at a high rate, in the step (B), allowing the cell to contact with a cancer antigen in a patient's body, an advantage that the CTL having an ability to kill the cancer cells specifically in the patient is induced is provided. Further, the cell population which contains a naive T cell or a naive T-like cell at a high rate can live in the patient's body for a long term. Although there is no particular limitation to a means for obtaining the cell population which contains naive T cells or naive T-like cells at a high rate, it is preferred, for example, to culture a material containing lymphocytes or lymphocyte progenitor cells in the presence of the above-mentioned fibronectin, a fibronectin fragment or a mixture thereof. In addition, as the cell population that contains naive T cells at a high rate, a cell population containing naive T cells at a high rate, which has been separated by a known method using the above-mentioned surface antigen marker of the naive T cells as an index, can be used.

As the cell population containing the lymphocytes, a cell population which contains a cancer cell-specific CTL at a high rate can also be used. As the cell population containing the cancer cell-specific cytotoxic T cells at a high rate, a cell obtained by the above-mentioned artificial cell culture procedure with use of, for example, peripheral blood mononuclear cells collected from a patient after the practice of the step (A) or after the practice of the step (B) can be used. As mentioned above, since a state where CTL having a cancer antigen-specific cytotoxicity is easily induced is realized after the practice of the step (A) and thus the cancer antigen-specific cytotoxic CTL is abundantly contained in the peripheral blood at a high proportion, such a material is suitable as a culture material of lymphocytes to be administered to a patient. In addition, as mentioned later, in the case where the set the step (A) and the step (B) are carried out several times, peripheral blood mononuclear cells are collected from a patient after the practice of the step (B), and they can be used in the lymphocyte administration of the step (B) in the next set of the steps. Moreover, as the cell population containing cancer cell-specific cytotoxic T cells at a high rate, tumor infiltrating lymphocytes (TIL) collected from malignant pleural effusions or neighboring lymph nodes or a culture thereof are exemplified as well.

The step (B) is carried out promptly in a patient after the practice of the step (A). Here, the “carried out promptly” includes carrying out the step (B) at an appropriate interval after the step (A) as long as a desired effect can be obtained by the administration of the lymphocytes in the step (B). For example, in the case where an anticancer agent is administered as the step (A), the interval can be appropriately set within the range where the reduction in lymphocytes is induced in consideration of the disposition of the anticancer agent used, e.g. the half-life in the blood, etc. The interval between the step (A) and the step (B) is, for example, one hour to 10 days, preferably 3 hours to 8 days, and more preferably 12 hours to 6 days. In addition, the interval between the step (A) and the step (B) in the case where radiation is performed in the step (A) may also be similarly set. The radiation dosage of the radioactive rays may be appropriately selected according to the usual therapy. Further, as mentioned later, in the case where the step (A) is performed several times and the step (B) is then performed, the interval can be calculated from the last time of the step (A).

In addition, the timing of the step (B) may be determined by confirming the lymphocyte count in the patient's blood with monitoring the state of the reduction in lymphocytes after the step (A). Although the present invention is not particularly limited to the above-mentioned aspect, the lymphocytes are to be administered, for example, at the time when the blood lymphocyte count of a patient is decreased to 1,000/μL or less for adults and 3,000/μL or less for children. Moreover, the present invention may also be performed by assuming the reduction in the lymphocytes through measurement of, for example, the blood neutrophil count and leukocyte count as an index of the reduction in the lymphocytes. Although the present invention is not particularly limited to the above-mentioned aspect, the step (B) is performed, for example, at the time when the neutrophil count in the patient's blood is decreased to 1,500/μL or less or the leukocyte count in the patient's blood is decreased to 4,000/μL or less.

In the treatment method of the present invention, an ingredient which can function as a vaccine for the cancer to be treated, i.e. a cancer vaccine may be administered. For example, a tumor antigen, a cell with an ability to present an antigen, an antigen-presenting cell, a tumor tissue-derived cell whose proliferation ability has been lost by artificial procedure, and an extract from a tumor tissue, etc. may also be administered.

Moreover, in the treatment method of the present invention, a lymphocyte stimulating factor such as an anti-CD3 antibody, an anti-CD28 antibody, a cytokine (IL-2, IL-15, IL-7, IL-12, IFN-γ, IFN-α, IFN-β, etc.), a chemokine, etc. may also be appropriately administered. In addition, in the present specification, the lymphocyte stimulating factor includes a lymphocyte growth factor.

It is preferable to carry out the administration of the cancer vaccine or the lymphocyte stimulating factor to a patient simultaneously with or after the administration of the lymphocytes in the step (B), whereby activation of the lymphocytes which have been administered from outside of the body will occur.

Furthermore, a method of treating cancer which includes repeating the combination of both the steps (A) and (B) two or more times is exemplified as a preferred aspect of the present invention. For example, such a method includes collecting peripheral blood mononuclear cells from a patient before carrying out the step (A), preparing a cell population containing lymphocytes by the above-mentioned known culture method using the peripheral blood mononuclear cells as the material, and performing the step (B) at an appropriate time using the cell population. Such a set of the steps (A) and (B) can be carried out two or more times. In the step (B) in the second set or later sets, it is possible to administer a cell population containing cancer cell-specific cytotoxic T cell at a higher rate by using a cell population which is obtained by the above-mentioned known method for culturing a lymphocyte collected from a patient after carry out the step (A) in the previous course or after the step (B), for example, peripheral blood mononuclear cells as the material.

Cancer to which a treatment method of the present invention is applied is not particularly limited. Examples of cancer include esophagus cancer, lung cancer, myeloma, ovarian cancer, head and neck cancer, and the like. As mentioned above, since a cancer vaccine may be used in combination in the present invention, the treatment method of the present invention is suitable for treating cancer which has been known to have a suitable vaccine, for example, cancer expressing a tumor antigen such as MAGE-A4, NY-ESO-1, SAGE, WT-1, MAGE-A3, gp100, or MART-1. In addition, the treatment method of the present invention may be applied to an anticancer agent therapy performed after the excision of the tumor tissue.

According to the present invention, a cancer immunity reconstruction therapy using human lymphocytes is provided to target intractable diseases, particularly myeloma, esophagus cancer, head and neck cancer, ovarian cancer, etc. The cancer immunity reconstruction therapy of the present invention includes a cancer therapy through treatment with a strong cytotoxicity (e.g. administration of anticancer agents and radiation) in combination with administration of cultured lymphocytes as an adoptive immunotherapy. Cancer cells are killed by the chemical and physical cancer therapy to reduce the size of cancer, and the cancer immunity in addition to the general immune function is augmented by administration of the lymphocytes. Since the lymphocytes are immediately supplemented to the patient after treatment with an anticancer agent or after radiation in the cancer immunity reconstruction therapy of the present invention, the number of the lymphocytes enough to maintain the immune function is maintained. As a result, the risk of the infectious diseases due to viruses or pathogenic microorganisms such as bacteria or fungi is greatly decreased.

The lymphocytes administered are made to contact with a tumor antigen which has been released as a result of destruction of cancer cells with an anticancer agent, etc., or a tumor antigen present on macrophages or dendritic cells having a high viability against anticancer agents, thereby highly inducing a specific cytotoxicity against cancers to be treated. Further, since the number of suppressor lymphocyte are reduced by anticancer agents, etc., activation of the cytotoxic lymphocytes is promoted. In addition, when the number of the lymphocytes has been restored, cancer immunity can be promoted more by administering the cancer vaccine.

By administering an anticancer agent, the tumor size is reduced together with the decrease of blood cells such as lymphocytes as the side effect due to the anticancer agent. In another aspect of the cancer immunity reconstruction therapy of the present invention, lymphocytes, preferably autologous lymphocytes which have been expanded, are given back to the patient to make it possible to prevent an infectious disease before the reduced lymphocytes increasingly cause various kinds of infectious diseases. In addition, this method makes it possible to prevent a decrease in the immune function usually seen in the cancer patients, and to improve patient's QOL (Quality of Life).

The therapeutic agent of the present invention will be explained in the following. The present invention provides a therapeutic agent for use in the cancer therapy according to the invention. The therapeutic agent of the present invention is characterized by containing lymphocytes as an active ingredient. In addition, said therapeutic agent is a therapeutic agent for cancer to be administered to the patient promptly after a treatment with an ability to induce the reduction in lymphocytes has been applied to the patient. Although the present invention is not particularly limited, the present invention encompasses a lymphocyte-containing formulation appended with a instruction for use in the treatment method of the present invention.

The lymphocyte as an active ingredient of the therapeutic agent of the present invention means a lymphocyte or a lymphocyte-containing cell population to be administered to a patient in the step (B) of the method of treating cancer according to the present invention. As mentioned above, the lymphocyte or the lymphocyte-containing cell population may be any one of an autologous lymphocyte derived from a patient, a donor lymphocyte collected from a donor other than the patient, and a culture obtained by an artificial cell culture procedure.

The therapeutic agent of the present invention can be formulated into a drop infusion or an injection by mixing, as an active ingredient, a lymphocyte, a lymphocyte-containing cell population, or a culture containing the lymphocyte or the cell population, with an organic or inorganic carrier, an excipient, a stabilizer, etc. which are known to be suitable for parenteral administration. The mode of administration is not particularly limited, and a means similar to a known medicine containing a cell (e.g. intravenous administration via injection or drop infusion) may be utilized.

Moreover, the present invention provides a cancer treatment kit containing an anticancer agent which causes the reduction in lymphocytes and a therapeutic agent separately. Examples of the anticancer agent which causes the reduction in lymphocytes include those used in the above-mentioned treatment method of the present invention and is used in the treatment of the step (A).

In addition, another aspect of the cancer treatment kit according to the present invention provides a cancer treatment kit including separately the therapeutic agent or the cancer treatment kit, and the cancer vaccine and/or the lymphocyte stimulating factor.

Another aspect of the cancer treatment kit according to the present invention provides a kit including a combination of an anticancer agent to be used in the treatment of the step (A) for cancer therapy of the present invention with an equipment for use in collection and culture of a lymphocyte to be administered in the step (B). Here, examples of the above equipment include, but not particularly limited to, a bag for collecting the blood, a container for cell culture (e.g. flask, bag, etc.) or other equipments. For example, a container coated with an anti-CD3 antibody and/or the above fibronectin fragment or a carrier (e.g. beads, etc.) coated with the above components are especially suitable for use in culturing the lymphocytes used in the present invention.

In addition, the present invention includes use of a lymphocyte or an anticancer agent in the production of the therapeutic agent for cancer of the present invention, use of an anticancer agent and lymphocytes in the production of a cancer treatment kit of the present invention, and use of lymphocytes in the method for cancer therapy of the present invention. For example, the therapeutic agent for cancer according to the present invention can be produced and provided by using patient's own cultured lymphocytes and an anticancer agent which causes the reduction in lymphocytes. Moreover, the cancer treatment kit of the present invention can be produced by using an anticancer agent which causes the reduction in lymphocytes and patient's own cultured lymphocytes as constituent ingredients. Moreover, the present invention encompasses use of an anticancer agent which causes the reduction in lymphocytes, a lymphocyte, a cancer vaccine and/or a lymphocyte stimulating factor in the production of a cancer treatment kit. For example, a cancer treatment kit of the fourth invention according to the present invention can be produced and provided by using patient's own cultured lymphocytes and a cancer vaccine and/or a lymphocyte stimulating factor as constituent ingredients of the cancer treatment kit of the present invention.

According to the present invention, in the expansion of the lymphocytes derived from a cancer patient with the depressed immune function, a high rate of the expansion of the lymphocytes can be obtained by using the culture equipment on which a CD3 ligand (such as anti-CD3 antibody) and a fibronectin (selected from fibronectin, a fibronectin fragment, or a mixture thereof) such as a human CH-296 fragment [polypeptide including the amino acid sequence as shown in SEQ ID NO: 13 of Sequence Listings, RetroNectin (registered trademark); manufactured by Takara Bio Inc.: hereinafter simply referred to as CH-296] are immobilized, regardless of the patient's cancer type even if the culture period is short. In addition, even if the culture is carried out under a condition of the plasma concentration lower than the general plasma concentration (1 to 10%) used in the case of lymphocyte culture, the expansion rate is not greatly reduced.

According to the present invention, a CCR7⁺CD45RA⁺ cell population, a CD27⁺CD45RA⁺ cell population, a CD28⁺CD45RA⁺ cell population, a CD62L⁺CD45RA⁺ cell population, and a CCR7⁺CD62L⁺CD45RA⁺ cell population can be obtained from the patient's PBMC regardless of the cancer type of the patient by using the culture equipment on which a CD3 ligand (such as the anti-CD3 antibody) and a fibronectin (such as CH-296) are immobilized in the lymphocyte expansion. These cellular phenotypes are all phenotypes which can be seen in naive T-like cells. The naive T-like cells are an index of the cells which are able to obtain a high therapeutic effect against cancer when they are given back to the body, such as accumulation in lymph nodes, high viability in the body, differentiation to the cell to show a high cytotoxicity against the cancer cells from cancer patients. That is, according to the present invention, use of the cancer patient's PBMC makes it possible to produce and provide a cell population of the highly expanded naive T-like cells having a high therapeutic effect against the cancer. In addition, in the expansion of the lymphocyte from the cancer patient's PBMC in the present invention, the cells obtained by using the culture equipment immobilized with a CD3 ligand and a fibronectin are those showing a remarkably high cytotoxicity against cancers and are useful in cancer therapy. Moreover, in the expansion of the lymphocyte derived from a cancer patient or a donor, a high expansion rate of the lymphocyte can be stably obtained with use of the culture equipment immobilized with a CD3 ligand and a fibronectin.

Incidentally, in the expansion of lymphocytes, in the case where a culture equipment immobilized with a CD3 ligand and a fibronectin is used, lymphocytes containing the naive T-like cell population at a higher expansion rate can be obtained compared to the case where a culture equipment immobilized with the CD3 ligand alone is used, and particularly the rate of the CCR7⁺ cell becomes extremely higher. CCR7 is known as a receptor of CCL21 which is a chemokine in the lymph nodes, and CCR7 expression cells may be expected to recognize an antigen in the lymph nodes and to differentiate to cytotoxicic lymphocytes. Thus, according to the present invention, a cell population wherein CCR7⁺ cell that can acquire a high ability to recognize the cancer cell in vivo from a patient and to attack the cancer cells can be produced with a high therapeutic effect against cancer.

In addition, when the lymphocyte obtained by expansion is subjected to an allogeneic mixed lymphocyte reaction (MLR) in the presence of a non-autologous cell, using a culture equipment immobilized with a CD3 ligand and a fibronectin, it has an excellent effect that the growth rate of cells for non-autologous antigen recognition is higher, compared to the lymphocyte obtained by using a culture equipment immobilized with a CD3 ligand alone. The cell shows high antigen recognition ability to the non-self antigen and makes it possible to bring about a higher therapeutic effect because it exhibits a non-self antigen-specific growth ability.

Since the concentration of an anticancer agent showing 50% inhibition of the cancer cell growth (50% growth inhibitory concentration; hereinafter referred to as GI₅₀) for the lymphocytes obtained with use of the culture equipment immobilized with the CD3 ligand and the fibronectin is higher than the remaining concentration in the blood, which has been generally reported, of each anticancer agent after administration, the lymphocytes show a strong proliferating property even in the presence of various anticancer agents. In addition, the above-mentioned GI₅₀ is higher than that obtained by using the culture equipment immobilized with the CD3 ligand alone. Thus, a cell population to which anticancer agent resistance is imparted is produced and provided by the present invention. For example, with use of the cancer patient's PBMC, a cell population showing resistance to anticancer agents can be obtained, and an adoptive immunotherapy using the cell population is extremely effective for treatment of cancer in combination with anticancer agents.

In addition, impartment of the anticancer agent resistance to the lymphocytes according to the present invention can be applied to any of lymphocytes derived from cancer patients and lymphocytes derived from donors, and such a method is extremely useful particularly for expansion of a cancer patient-derived lymphocyte having reduced biological activity.

The lymphocytes obtained by expansion with use of the culture equipment immobilized with the CD3 ligand and the fibronectin can be fractionated so that the rate of the naive T-like cells is raised, and the T cell rate and the T cell recovery rate after administration of the cell become much higher by combinatorial administration of an anticancer agent such as mitomycin C (MMC). This demonstrates that the survival rate of naive T-like cells in the living body is high and administration of the expanded naive T-like cells can restore early the decrease of the lymphocyte count due to the anticancer agent. Thus, an adoptive immunotherapy using the cell population wherein the naive T-like cells are highly effectively proliferated is extremely effective for the treatment of cancer in combination with an anticancer agent.

According to the present invention, a cellular medicine resistant to anticancer agents can be provided, which can be used in combination with an anticancer agent. An aspect of the cellular medicine includes, as an active ingredient, a cell to which an anticancer agent resistance is imparted. These cellular medicines can be administered in the state where an anticancer agent administered remains in the body, and the cellular medicines can show an effect in the presence of an anticancer agent, i.e. in the state where the anticancer agent still remains. These cellular medicines can be prepared as lymphocytes derived from a cancer patient having the suppressed immunity, and can be used as a cellular medicine derived from a cancer patient to which anticancer resistance is imparted, in such a condition that the anticancer agent remains.

In accordance with the present invention, a method including the step of culturing a lymphocyte in the presence of a CD3 ligand and a fibronectin for imparting anticancer resistance to a lymphocyte derived from a cancer patient or a donor can be provided, and such a method is useful for an adoptive immunotherapy to cancer patients.

Another aspect of the present invention includes use of lymphocytes expanded under such a condition that the anticancer agent remains, which are derived from a cancer patient and to which anticancer agent resistance is imparted; use of expanded lymphocytes in the production a therapeutic agent for cancer, which are derived from a cancer patient and to which anticancer agent resistance is imparted; and a method of treating cancer using lymphocytes which are derived from a cancer patient and to which anticancer agent resistance is imparted.

Moreover, according to the present invention, a kit with anticancer agent resistance to a lymphocyte derived from a cancer patient, including a CD3 ligand and a fibronectin, and a production method of a cancer patient-derived lymphocyte to which anticancer agent resistance is imparted, including the step of culturing a lymphocyte in the presence of a CD3 ligand and a fibronectin can be provided.

### EXAMPLES

The invention will be more specifically described hereinafter by way of examples, without intending to limit the scope of the present invention thereto.

### Example 1

### Studies on Effects of Transferred T Cells after Administration of Anticancer Agent Using Syngeneic Mouse Tumor Model

### (1) Preparation of Highly Metastatic B16F10 Cells

A mouse melanoma B16F10 cell line (provided by Institute of Development, Aging and Cancer, Tohoku University) is administered to 7-week old female C57BL/6 mice (available from Japan SLC, Inc.) via a tail vein under anesthesia, and lung metastasis tumor is collected 14 days later. The collected tumor is dispersed into a single cell, and the resulting cells are cultured in vitro, then administered to mice again. This procedure is repeated three times. When 1×10⁵ cells are finally administered, highly metastatic B16F10 cells (hereinafter referred to as hB16F10) showing lung metastasis of about 50 to 100 cells after 14 days of culture are obtained.

(2) Preparation of Splenic Lymphocytes from Cancer-Bearing Mouse

The hB16F10 is suspended in Dulbecco's phosphate buffered saline (manufactured by Baxter International Inc., Sigma Corp., or Invitrogen Corp., hereinafter referred to as DPBS) to a density of 5×10⁵ cells/mL. The cell suspension (0.2 mL) is administered to 7-week old female C57BL/6 mice via a tail vein under anesthesia, thereby developing a lung metastasis tumor. After 14 days, the spleen is extracted and homogenized with use of a glass slide in an RPMI 1640 medium (manufactured by Sigma). The homogenized spleens from 10 mice are put together using the RPMI 1640 medium and collected into a tube to make the volume 45 mL. The tube is allowed to stand on ice for 5 minutes, and transferred to a fresh tube through a 40 μm cell strainer (manufactured by Beckton Dickinson Corporation). The supernatant after centrifugation is removed, and the precipitate is suspended in 2 mL of an ACK buffer (0.15 M NH₄Cl, 0.01 M KHCO₃, 0.01 mM Na₂EDTA, pH 7.4) for hemolysis procedure. An ACK buffer (2 mL) is further added to this suspension to form a suspension, and an RPMI 1640 medium is added to make the volume of the cell suspension 50 mL. The supernatant after centrifugation is removed, and the cells are suspended in 10 mL of an RPMI 1640 medium, followed by transferring the suspension into a fresh tube through a cell strainer. After addition of the RPMI 1640 medium to the cell suspension so as to make up a volume of 40 mL, the mixture is centrifuged to remove the supernatant, and the cells are suspended in an equivalent mixture of an RPMI 1640 medium and CP-1 (manufactured by Kyokuto Pharmaceutical Industrial Co., Ltd.) containing 8% human serum albumin (HSA, drug name; BUMINATE; manufactured by Baxter International Inc.), and then stored in liquid nitrogen until its use.

### (3) Immobilization of Anti-Mouse CD3 Antibody and Human CH-296 Fragment

An anti-mouse CD3 antibody and a human CH-296 fragment [a polypeptide comprising the amino acid sequence shown in SEQ ID NO 13 of Sequence Listings, RetroNectin (registered trademark); manufactured by Takara Bio Inc.: hereinafter simply referred to as CH-296] are immobilized to a culture equipment which is used in the following experiment. In other words, ACD-A solution (manufactured by Terumo Corporation) containing an anti-mouse CD3 antibody (manufactured by R&D Systems Inc.) (final concentration: 7 μg/mL) is added in an amount of 800 μL/well each to a 12-well cell culture plate (manufactured by Corning Inc.), and incubation is carried out at 4° C. overnight. Thereafter, in the CH-296 stimulation group, a human CH-296 is added to a final concentration of 25 μg/mL and incubation is further carried out at room temperature for 5 hours. After removal of the ACD-A solution containing the antibody/CH-296 by aspiration from the culture equipment just before use, each well is washed twice with DPBS and once with an RPMI 1640 medium, and then subjected to the experiment.

(4) Purification of Splenic Lymphocytes with Nylon Fibers

The splenic lymphocytes prepared in item (2) of Example 1 are purified with use of nylon fibers in order to increase the purity of the lymphocytes. A 10 mL-syringe (manufactured by Terumo Corporation) is filled up with 0.6 g of the nylon fibers (manufactured by Wako Pure Chemical Industries, Ltd.), equilibrated with DPBS, and sterilized at 121° C. for 20 minutes. The column is equilibrated with an RPMI 1640 medium containing 10% fetal bovine serum (manufactured by MP Biomedicals, LLC; hereinafter referred to as FBS) and incubated in a 5% CO₂ incubator at 37° C. for one hour. The splenic lymphocytes prepared in item (2) of Example 1 are suspended in an RPMI 1640 medium (2 to 3 mL) containing 10% FBS so as not to exceed 2×10⁸ cells, applied to the column, and incubated in a 5% CO₂ incubator at 37° C. for one hour. The 10% FBS-containing an RPMI 1640 medium (15 mL) which has been previously warmed at 37° C. is added to the column and the eluted cells are collected.

### (5) Expansion of Mouse T Cell Populations

The lymphocytes prepared in item (4) of Example 1 are suspended in GT-T503 medium (manufactured by Takara Bio Inc.) (hereinafter referred to as a culture medium) containing 10% FBS, 0.1 mM NEAA mixture (manufactured by Cambrex Corporation), 1 mM sodium pyruvate (manufactured by Cambrex Corporation), 50 μM 2-mercaptoethanol (manufactured by Nacalai Tesque, Inc.), and 0.2% HSA so as to have a density of 1.5×10⁶ cells/mL. The culture medium is previously added to a plate immobilized with the anti-mouse CD3 antibody or the anti-mouse CD3 antibody and the human CH-296 prepared in item (3) of Example 1 in a volume of 1.4 mL/well, the above cell suspension is added thereto in a volume of 1 mL/well each, and these plates are incubated at 37° C. in a 5% CO₂ incubator (on day 0 of culture). On day 3 of culture, the cell suspension is diluted using the culture medium so as to have a density of 1.5×10⁵ cells/mL, and the whole is transferred to a fresh 175 cm²-cell culture flask (manufactured by Corning Inc.) to which nothing is immobilized. At this time, mouse IL-2 (manufactured by R&D Systems Inc.) was added so as to have a final concentration of 100 U/mL, and mouse IL-7 (manufactured by R&D Systems Inc.) was added so as to have a final concentration of 10 ng/mL. On day 7 of culture, the cells were collected, and subjected to a test with the following syngeneic tumor model.

(6) Evaluation of Transferred T Cell Population in Syngeneic Tumor Model of C57BL/6-hB16F10 after Administration of Anticancer Agent

The hB16F10 is administered to 7-week old female C57BL/6 mice from a tail vein under anesthesia in the same manner as in item (2) of Example 1. On day 3 after the administration, cisplatin (manufactured by Nichi-Iko Pharmaceutical Co., Ltd.) and mitomycin C (manufactured by Kyowa Medex Co., Ltd., hereinafter referred to as MMC) are intraperitoneally administered as anticancer agents at a dose of 5 mg/kg and 2 mg/kg in the cisplatin administration group and the mitomycin C administration group, respectively. Another two days later, the cells prepared in item (4) of Example 1 or the cells prepared in item (5) of Example 1 are suspended in DPBS so as to have a respective density of 2.5×10⁸ cells/mL, and 0.2 mL of the suspension is administered via a tail vein. A group without administration of the cells is set as a control group. The number of the lymphocytes in the peripheral blood is regularly measured until 14 days after administration of the cells. In addition, the number of the lung metastasis cells is counted on the final day (on day 14 after the cell administration) by subjecting the mice to exsanguinations after anesthesia, excising the lung, and counting the metastatic colonies. As a result, in the cell administration group, it is thought that a preventive effect due to large lymphocyte counts of the peripheral blood in all mice can be shown against infectious disease associated with the reduction in number of the lymphocytes by administration of anticancer agents. In addition, in the cell administration group, a combination effect with the anticancer agent can be confirmed because the number of the lung metastasis is fewer in all mice than that of the administration group of the anticancer agent alone.

### Example 2

### Expansion of Lymphocytes from Peripheral Blood Mononuclear Cell (PBMC) of Cancer Patient-1

### (1) Separation of PBMC and Inactivated Plasma

Heparin-added blood sample was collected in a volume of 50 to 58 mL from a donor of a human cancer patient with informed consent, and the obtained blood was centrifuged at 700×g for 20 minutes. After the centrifugation, the supernatant plasma fraction and the PBMC-containing cell fraction were respectively collected. The plasma fraction was inactivated at 56° C. for 30 minutes and centrifuged at 900×g for 30 minutes. The supernatant after the centrifugation was collected as an inactivated plasma and subjected to each experiment. The PBMC-containing cell fraction was diluted with DPBS and overlaid on Ficoll-paque (manufactured by GE Healthcare Bio-Sciences), and then centrifuged at 700×g for 20 minutes. The intermediate layer of the PBMC was collected with a pipette, washed, and the viable cell count was calculated using an automated blood cell counting device (NucleoCounter; manufactured by ChemoMetec A/S) and the cells were then subjected to each experiment.

### (2) Immobilization of Anti-CD3 Monoclonal Antibody (OKT3) and CH-296

The OKT3 and the CH-296 were immobilized to the culture equipment used in the following experiment. That is, an ACD-A solution containing the OKT3 (final concentration: 5 μg/mL) and the CH-296 (final concentration: 25 μg/mL) was added respectively to a gas-permeable culture bag CultiLife 215 (manufactured by Takara Bio, Inc.) in 10.4 mL/bag (in the case of the area: 86 cm² at the start of culture) or 26.0 mL/bag (in the case of the area: 215 cm² at the start of culture), and incubated at 37° C. for 5 hours in a 5% CO₂ incubator. The above bag was washed three times with the RPMI 1640 medium before use and then subjected to each experiment.

### (3) Expansion of Lymphocytes

The PBMCs of 0.7×10⁷ to 1.2×10⁷ cells prepared in item (1) of Example 2 were suspended in 120 mL (in the case of the area of 86 cm² at the start of culture) of KBM551 (manufactured by Takara Bio Inc.; hereinafter referred to as plasma-containing KBM551) containing a 0.6 to 1.0% inactivated plasma, or the PBMCs of 2.1×10⁷ to 4.2×10⁷ cells were suspended in 300 mL (in the case of the area of 215 cm² at the start of culture) of KBM551 containing a 0.6 to 1.0% inactivated plasma, and added to CultiLife 215 immobilized with the OKT3 and the CH-296 prepared in item (2) of Example 2. IL-2 (Drug name: Proleukin; manufactured by Chiron) was added to the suspension so as to have a final concentration of 200 U/mL and the mixture was incubated at 37° C. under 5% CO₂ (on day 0 of culture). On day 4 of culture, the cell solution in each CultiLife 215 was suspended, and a part of the suspension was diluted and transferred to a gas permeable culture bag CultiLifeEva (manufactured by Takara Bio Inc.) on which nothing was immobilized. On this occasion, the cell solution was added in 9.4 mL per culture area of 100 cm², and the plasma-containing KBM551 was added thereto in 68.8 mL per culture area of 100 cm². After addition of IL-2 so as to have a final concentration of 200 U/mL, the solution was incubated at 37° C. under 5% CO₂. The solution was continued to effect the culturing, diluted two-fold by the addition of an equivalent amount of plasma-free KBM551 (hereinafter referred to as plasma-free KBM551) to each CultiLifeEva on day 7 of culture, and added with IL-2 to a final concentration of 200 U/mL each. On day 10 of culture, after the cell solution in each CultiLifeEva was suspended, a half of the suspension was removed, and the solution was diluted two fold by the addition of an equivalent amount of plasma free KBM551 relative to the cell solution, and added with IL-2 to a final concentration of 200 U/mL in all cases. On days 10 and 14 of culture, the viable cell count was counted using an automated blood cell counting device to calculate the expansion rate in comparison with the cell count at the start of culture. The results are shown in Table 1.

TABLE 1

| | | Expansion Fold (Fold) | |
|---|---|---|---|
| Cancer | | On Day 10 of | On Day 14 of |
| Patient No. | Cancer Type | Culture | Culture |
| PT001 | Gastric Cancer | x431 | N.T. |
| PT002 | Breast Cancer | x554 | N.T. |
| PT003 | Breast Cancer | x358 | N.T. |
| PT004 | Pancreatic | x319 | N.T. |
| | Cancer | | |
| PT005 | Pancreatic | x466 | N.T. |
| | Cancer | | |
| PT006 | Breast Cancer | x610 | N.T. |
| PT007 | Esophageal | x290 | x385 |
| | Cancer | | |
| PT008 | Laryngeal | x338 | x646 |
| | Cancer | | |
| PT009 | Colon Cancer | x471 | x1013 |
| PT010 | Stomach Cancer | x336 | N.T. |
| PT011 | Breast Cancer | x463 | N.T. |
| PT012 | Rectal Cancer | x435 | x1036 |
| PT013 | Breast Cancer | x796 | x1484 |

N.T. = No Test

As shown in Table 1, as a result of using the culture equipment immobilized with the anti-CD3 antibody and the CH-296 in the expansion of the lymphocytes derived from a cancer patient with the reduced immunity and lymphocyte activity, an expansion fold of the lymphocytes was high in any type of cancers at any stage of culture period. In addition, it was elucidated that the expansion fold of the lymphocytes was high even under the condition that the plasma concentration was lower than the concentration (1 to 10%) which had been generally used in the culture of the lymphocytes.

(4) Analysis of CCR7⁺CD45RA⁺ Cell, CD27⁺CD45RA⁺ Cell, CD28⁺CD45RA⁺ Cell, CD62L⁺CD45RA⁺ Cell, and CCR7⁺CD62L⁺CD45RA⁺ Cell (⁺ Shows Having Positive Antibody Reactivity)

The cells on days 10 and 14 of culture, prepared in item (3) of Example 2, were washed with DPBS containing 1% bovine serum albumin (BSA, manufactured by Sigma) (hereinafter referred to as 1% BSA/DPBS) or DPBS, and the cells were suspended in 1% BSA/DPBS, then FITC labeled mouse IgG1/RD1 labeled mouse IgG1/PC5 labeled mouse IgG1 (manufactured by Beckman Coulter Inc.) were added thereto as a negative control. Similarly, cells added with RD1 labeled mouse anti-human CD45RA antibody (manufactured by Beckman Coulter Inc.)/FITC labeled mouse anti-human CCR7 antibody (manufactured by R&D Systems), cells added with RD1 labeled mouse anti-human CD45RA antibody/FITC labeled mouse anti-human CD28 antibody (manufactured by eBioscience, Inc.)/PC5 labeled mouse anti-human CD27 antibody (manufactured by Beckman Coulter Inc.), and cells added with RD1 labeled mouse anti-human CD45RA antibody/FITC labeled mouse anti-human CCR7 antibody/PC5 labeled mouse anti-human CD62L antibody (manufactured by Beckman Coulter Inc.) were prepared. After addition of each antibody, incubation was carried out on ice for 30 minutes. After the incubation, the cells were washed with DPBS containing 0.1% BSA (hereinafter referred to as 0.1% BSA/DPBS), and suspended again in DPBS. The ratio of CCR7⁺CD45RA⁺ cells, CD27⁺CD45RA⁺ cells, CD28⁺CD45RA⁺ cells, CD62L⁺CD45RA⁺ cells, and CCR7⁺CD62L⁺CD45RA⁺ cells of each cell population was calculated. The results are shown in Table 2, Table 3, Table 4, Table 5, and Table 6.

TABLE 2

| | |
|---|---|
| Cancer | CCR7⁺CD45RA⁺ (%) |

| Patient | Cancer | On Day 10 | On day 14 |
|---|---|---|---|
| No. | Type | of culture | of culture |
| PT001 | Gastric cancer | 13.4 | N.T. |
| PT002 | Breast cancer | 16.9 | N.T. |
| PT003 | Breast cancer | 8.1 | N.T. |
| PT004 | Pancreatic cancer | 39.7 | N.T. |
| PT005 | Pancreatic cancer | 18.7 | N.T. |
| PT006 | Breast cancer | 44.3 | N.T. |
| PT007 | Esophageal cancer | 13.6 | 12.5 |
| PT008 | Laryngeal cancer | 4.2 | 9.5 |
| PT009 | Colon cancer | 23.4 | 8.0 |
| PT0010 | Stomach cancer | 19.4 | N.T. |
| PT0011 | Breast cancer | 4.7 | N.T. |
| PT0012 | Rectal cancer | 16.4 | 12.2 |
| PT0013 | Breast cancer | 37.3 | 42.8 |

TABLE 3

| | |
|---|---|
| Cancer | CD27⁺CD45RA⁺ (%) |

| Patient | | On Day 10 | On day 14 |
|---|---|---|---|
| No. | Cancer Type | of culture | of culture |
| PT001 | Gastric cancer | 47.0 | N.T. |
| PT002 | Breast cancer | 58.2 | N.T. |
| PT003 | Breast cancer | 35.1 | N.T. |
| PT004 | Pancreatic cancer | 56.3 | N.T. |
| PT005 | Pancreatic cancer | 49.8 | N.T. |
| PT006 | Breast cancer | 80.8 | N.T. |
| PT007 | Esophageal cancer | 33.8 | 29.0 |
| PT008 | Laryngeal cancer | 32.3 | 31.7 |
| PT009 | Colon cancer | 24.6 | 24.5 |
| PT010 | Stomach cancer | 50.4 | N.T. |
| PT011 | Breast cancer | 26.5 | N.T. |
| PT012 | Rectal cancer | 44.3 | 25.7 |
| PT013 | Breast cancer | N.T. | 63.8 |

TABLE 4

| | |
|---|---|
| Cancer | D28⁺CD45RA⁺ (%) |

| Patient | | On Day 10 | On day 14 |
|---|---|---|---|
| No. | Cancer Type | of culture | of culture |
| PT001 | Gastric cancer | 53.7 | N.T. |
| PT002 | Breast cancer | 61.1 | N.T. |
| PT003 | Breast cancer | 34.1 | N.T. |
| PT004 | Pancreatic cancer | 59.0 | N.T. |
| PT005 | Pancreatic cancer | 48.8 | N.T. |
| PT006 | Breast cancer | 82.8 | N.T. |
| PT007 | Esophageal cancer | 39.5 | 40.3 |
| PT008 | Laryngeal cancer | 49.4 | 52.6 |
| PT009 | Colon cancer | 24.5 | 36.0 |
| PT010 | Stomach cancer | 45.7 | N.T. |
| PT011 | Breast cancer | 32.9 | N.T. |
| PT012 | Rectal cancer | 41.8 | 23.9 |
| PT013 | Breast cancer | 86.6 | 65.2 |

TABLE 5

CD62L⁺CD45RA⁺ (%)

| Cancer | | On Day 10 | On day 14 |
|---|---|---|---|
| Patient No. | Cancer Type | of culture | of culture |
| PT006 | Breast cancer | 91.5 | N.T. |
| PT007 | Esophageal cancer | 29.0 | 48.4 |
| PT008 | Laryngeal cancer | 79.4 | 20.6 |
| PT009 | Colon cancer | 31.7 | 73.4 |
| PT012 | Rectal cancer | 37.7 | N.T. |
| PT013 | Breast cancer | 74.5 | N.T. |

TABLE 6

| | | CCR7⁺CD62L⁺CD45RA⁺ | |
|---|---|---|---|
| | | (%) | |
| Cancer | | On Day 10 | On day 14 |
| Patient No. | Cancer Type | of culture | of culture |
| PT006 | Breast cancer | 32.7 | N.T. |
| PT007 | Esophageal cancer | 46.4 | 14.7 |
| PT008 | Laryngeal cancer | 12.5 | 8.8 |
| PT009 | Colon cancer | 10.3 | 12.6 |
| PT012 | Rectal cancer | 9.8 | N.T. |
| PT013 | Breast cancer | 35.5 | N.T. |

As shown in Tables 2, 3, 4, 5, and 6, a CCR7⁺CD45RA⁺ cell population, a CD27⁺CD45RA⁺ cell population, a CD28⁺CD45RA⁺ cell population, a CD62L⁺CD45RA⁺ cell population, and a CCR7⁺CD62L⁺CD45RA⁺ cell population were obtained from the PBMCs of any cancer patients by using the culture equipment immobilized with the anti-CD3 antibody and the CH-296 in the expansion of the lymphocytes. These cells have all phenotypes typical of naive T-like cells, and a high therapeutic effect against cancer may be expected when the lymphocytes after the expansion are given back to the body because of the accumulation of the cells in the lymph node, the rise of the cell viability in the body, and the differentiation to the cell with a high cytotoxicity against the cancer cell derived from a cancer patient. It was elucidated from this example that a cell population having a high therapeutic effect against cancer, wherein naive T-like cells had been proliferated at high efficiency, can be produced by using a combination of the anti-CD3 antibody and the CH-296 in the expansion of the lymphocytes using PBMC of the cancer patient.

### (5) Determination of Non-Autologous Specific Cytotoxicity

Cytotoxicity of the cells on days 10 and 14 of culture, prepared in item (3) of Example 2, was assayed according to a cytotoxicity assay using Calcein-AM (Richtenfels R., et al., J. Immunol. Methods, vol. 172, No. 2, pp. 227-239 (1994)). K562 cells (ATCC CCL-243, hereinafter referred to as K562) and Daudi cells (ATCC CCL-213, hereinafter referred to as Daudi) were suspended in an RPMI 1640 medium containing 5% FBS so as to have a density of 1×10⁶ cells/mL, and Calcein-AM (manufactured by Dojindo Laboratories) was added to a final concentration of 25 μM, and then the mixture was incubated at 37° C. for one hour. The cells were washed with a Calcein-AM free medium to afford Calcein labeled target cells.

The cells as effector cells on days 10 and 14 of culture, derived from cancer patient No. PT008 and prepared in item (3) of Example 2, were serially diluted with an RPMI 1640 medium containing a 5% human AB type serum, 2 mM L-glutamine (all manufactured by Cambrex Corp.), 1 mM sodium pyruvate, 1×NEAA Mixture, 100 μg/mL streptomycin sulfate (manufactured by Meiji Seika Kaisha, Ltd.)(hereinafter referred to as 5HRPMI), so as to have a density of from 3×10⁵ to 9×10⁶ cells/mL. Thereafter, the dilution was previously dispensed to each well of a 96-well cell culture plate (manufactured by Beckton Dickinson Corporation or Corning Inc.) in a volume of 100 μL/well each, and the Calcein-labeled target cells (K562 or Daudi) in a volume of 100 μL/well were added to these plates so that it had a density of 1×10⁵/mL. At this time, the ratio of the effector cells (E) to the Calcein-labeled target cells (T) was expressed as an E/T ratio, and assays were carried out at the E/T ratios of 90, 30, 10, and 3. The plate containing the above cell suspension was centrifuged at 210×g for 1 minute, and thereafter the cells were incubated in the presence of 5% CO₂ at 37° C. for 4 hours. After 4 hours, 100 μL of the culture supernatant was collected from each well, and the amount of calcein released into the culture supernatant was determined with a fluorescence plate reader (manufactured by Berthold Technologies GmbH) (excited at 485 nm/measured at 538 nm). “Cytotoxicity (%)” was calculated in accordance with the following formula 1.

Cytotoxicity (%)={(Measured Value in Each Well−Minimum Released Amount)/(Maximum Released Amount−Minimum Released Amount)}×100 Formula 1:

In the above formula, the minimum released amount is an amount of calcein released in the well containing only calcein labeled target cells, showing an amount of calcein naturally released from the calcein-labeled target cells. In addition, the maximum released amount refers to an amount of calcein released when the cells are completely disrupted by adding 0.1% of a surfactant Triton X-100 (manufactured by Nakalai Tesque Inc.) to the cells. The results of the assay are shown in Table 7.

TABLE 7

| Cancer | | | | Cytotoxicity (%) | |
|---|---|---|---|---|---|
| Patient | Cancer | Target | | On Day 10 | On day 14 |
| No. | Type | Cells | E/T Ratio | of culture | of culture |
| PT008 | Laryngeal | K562 | 90 | 77.8 | 60.1 |
| | cancer | | | | |
| ″ | Laryngeal | ″ | 30 | 68.6 | 31.6 |
| | cancer | | | | |
| ″ | Laryngeal | ″ | 10 | 38.7 | 12.0 |
| | cancer | | | | |
| ″ | Laryngeal | ″ | 3 | 10.4 | 0.2 |
| | cancer | | | | |
| ″ | Laryngeal | Daudi | 90 | 68.9 | 54.8 |
| | cancer | | | | |
| ″ | Laryngeal | ″ | 30 | 59.0 | 40.1 |
| | cancer | | | | |
| ″ | Laryngeal | ″ | 10 | 45.6 | 32.7 |
| | cancer | | | | |
| ″ | Laryngeal | ″ | 3 | 15.8 | 17.6 |
| | cancer | | | | |

As shown in Table 7, the cells obtained by expansion of the lymphocytes derived from PBMCs of the cancer patients using the culture equipment immobilized with the anti-CD3 antibody and the CH-296 showed an extremely high cytotoxicity against cancer regardless of the culture period, and they were useful cells for cancer therapy.

### Example 3

### Expansion of Lymphocytes from PBMCs of Cancer Patient-2

### (1) Immobilization of OKT3 and CH-296

In a similar manner to item (2) of Example 2, the OKT3 and the CH-296 were immobilized to the culture equipment used in the following experiment.

### (2) Expansion of Lymphocytes

In a similar manner to item (3) of Example 2, the expansion of the lymphocytes was carried out, except that the basal medium used for culturing was GT-T503 containing 0.2% human HSA (hereinafter referred to as 0.2% HSA/GT-T503), the medium used at the start of culture and on day 4 of culture was 0.2% HSA/GT-T503 containing 0.6% autologous plasma derived from cancer patients, and the medium used on days 7 and 10 of culture was plasma-free 0.2% HSA/GT-T503. On day 10 of culture, the viable cell count was counted using an automated blood cell counting device to calculate the expansion fold in comparison with the cell count at the start of culture. The results are shown in Table 8.

TABLE 8

| Cancer | | |
|---|---|---|
| Patient | | Expansion Fold |
| No. | Cancer Type | (Fold) |
| PT001 | Gastric | x603 |
| | cancer | |
| PT002 | Breast cancer | x498 |
| PT003 | Breast cancer | x359 |

As shown in Table 8, regardless of the kind and property of the medium to be used for culture, a stable and high expansion rate of the lymphocytes was obtained with use of the culture equipment immobilized with the anti-CD3 antibody and the CH-296 in the expansion of the lymphocytes.

### (3) Analysis of CCR7⁺CD45RA⁺Cells, CD27⁺CD45RA⁺ Cells, and CD28⁺CD45RA⁺Cells

With respect to the cells on day 10 of culture, prepared in item (2) of Example 3, analysis of CCR7⁺CD45RA⁺ cells, CD27⁺CD45RA⁺ cells, and CD28⁺CD45RA⁺ cells was similarly performed according to the method described in item (4) of Example 2. The results are shown in Table 9, Table 10, and Table 11.

TABLE 9

| Cancer Patient No. | Cancer Type | CCR7⁺CD45RA⁺ (%) |
|---|---|---|
| PT001 | Gastric Cancer | 10.3 |
| PT002 | Breast Cancer | 22.0 |
| PT003 | Breast Cancer | 10.1 |

TABLE 10

| Cancer Patient No. | Cancer Type | CCR27⁺CD45RA⁺ (%) |
|---|---|---|
| PT001 | Gastric Cancer | 26.8 |
| PT002 | Breast Cancer | 33.5 |
| PT003 | Breast Cancer | 23.6 |

TABLE 11

| Cancer Patient No. | Cancer Type | CD28⁺CD45RA⁺ (%) |
|---|---|---|
| PT001 | Gastric Cancer | 27.5 |
| PT002 | Breast Cancer | 43.2 |
| PT003 | Breast Cancer | 26.5 |

As shown in Tables 9, 10 and 11, regardless of the kind and property of the medium to be used for culture, a CCR7⁺CD45RA⁺ cell population, a CD27⁺CD45RA⁺ cell population, and a CD28⁺CD45RA⁺ cell population were obtained from the PBMCs of patients with any type of cancer in the expansion of the cancer patient-derived lymphocytes by using the culture equipment immobilized with the anti-CD3 antibody and the CH-296. These cells have all phenotypes typical of naive T-like cells, and a high therapeutic effect on cancer may be expected when the lymphocytes after the expansion are given back to the body because of the accumulation of the cells in the lymph nodes, the rise of the cell viability in the body, and the differentiation to the cells with high cytotoxicity against the cancer cells derived from the cancer patient. It was elucidated from this example that a cell population having a high therapeutic effect against cancer, wherein naive T-like cells had been proliferated at high efficiency, can be produced by using a combination of the anti-CD3 antibody and the CH-296 in the expansion of the lymphocytes using PBMCs of the cancer patient.

### Example 4

### Expansion of Lymphocytes from PBMCs of Cancer Patient-3

### (1) Immobilization of OKT3 and CH-296

In a similar manner to item (2) of Example 2, the OKT3 and the CH-296 were immobilized to the culture equipment used in the following experiment.

### (2) Expansion of Lymphocytes

In a similar manner to item (3) of Example 2, the expansion of the lymphocytes derived from cancer patient No. PT006 was carried out, except that the autologous plasma concentration of the plasma-containing KBM551 used on days 0 and 4 of culture was 0.6% or 1.2%, and on day 7 of culture, the final plasma concentration was adjusted to the concentration as shown in Table 12, using a plasma free KBM551 or 0.6% plasma-containing KBM551. On day 10 of culture, the cell solution was added with IL-2 to a final concentration of 200 U/mL without dilution. The results are shown in Table 12.

TABLE 12

| | | Final Plasma Concentration in | | | | | Expansion Fold | |
|---|---|---|---|---|---|---|---|---|
| | | Culture Medium | | | | | (Fold) | |
| | | On Day | On Day | On Day | On Day | | On Day | On Day |
| | | 0 from | 4 from | 7 from | 10 from | | 10 from | 14 from |
| Cancer | | Start | Start | Start | Start | | Start | Start |
| Patient | Cancer | of | of | of | of | Days for | of | of |
| No. | Type | Culture | Culture | Culture | Culture | Culture | Culture | Culture |
| PT006 | Breast | 0.6% | 0.6% | 0.6% | 0.6% | 10 Days | ×640 | N.T. |
| | Cancer | | | | | | | |
| ″ | Breast | 1.2% | 1.2% | 0.6% | 0.6% | 14 Days | ×645 | ×795 |
| | Cancer | | | | | | | |

As shown in Table 12, regardless of the plasma concentration in the culture solution, a stable and high expansion rate of the lymphocytes was obtained with use of the culture equipment immobilized with the anti-CD3 antibody and the CH-296 in the expansion of the lymphocytes.

### (3) Analysis of CCR7⁺CD45RA⁺ Cells, CD27⁺CD45RA⁺ Cells, and CD28⁺CD45RA⁺ Cells

With respect to the cells on days 10 and 14 of culture, prepared in item (2) of Example 4, analysis of CCR7⁺CD45RA⁺ cells, CD27⁺CD45RA⁺ cells, and CD28⁺CD45RA⁺ cells was performed similarly to the method described in item (4) of Example 2. The results are shown in Tables 13, 14, and 15.

TABLE 13

| | | Final Plasma Concentration in | | | | | | |
|---|---|---|---|---|---|---|---|---|
| | | Culture Medium | | | | | CCR7⁺CD45RA⁺ (%) | |
| | | On Day | On Day | On Day | On Day | | On | |
| | | 0 from | 4 from | 7 from | 10 from | | Day | On Day |
| Cancer | | Start | Start | Start | Start | | 10 from | 14 from |
| Patient | Cancer | of | of | of | of | Days for | Start | Start |
| No. | Type | Culture | Culture | Culture | Culture | Culture | of Culture | of Culture |
| PT006 | Breast | 0.6% | 0.6% | 0.6% | 0.6% | 10 Days | 42.9 | N.T. |
| | Cancer | | | | | | | |
| ″ | Breast | 1.2% | 1.2% | 0.6% | 0.6% | 14 Days | 32.1 | 42.6 |
| | Cancer | | | | | | | |

TABLE 14

| | | Final Plasma Concentration in | | | | | | |
|---|---|---|---|---|---|---|---|---|
| | | Culture Medium | | | | | CD27⁺CD45RA⁺ (%) | |
| | | On Day | On Day | On Day | On Day | | | On |
| | | 0 from | 4 from | 7 from | 10 from | | On Day | Day |
| Cancer | | Start | Start | Start | Start | | 10 from | 14 from |
| Patient | Cancer | of | of | of | of | Days for | Start | Start |
| No. | Type | Culture | Culture | Culture | Culture | Culture | of Culture | of Culture |
| PT006 | Breast | 0.6% | 0.6% | 0.6% | 0.6% | 10 Days | 78.4 | N.T. |
| | Cancer | | | | | | | |
| ″ | Breast | 1.2% | 1.2% | 0.6% | 0.6% | 14 Days | 74.6 | 68.1 |
| | Cancer | | | | | | | |

TABLE 15

| | | Final Plasma Concentration in | | | | | CD28⁺CD45RA⁺ | |
|---|---|---|---|---|---|---|---|---|
| | | Culture Medium | | | | | (%) | |
| | | On Day | On Day | On Day | On Day | | On Day | On Day |
| | | 0 from | 4 from | 7 from | 10 from | | 10 from | 14 from |
| Cancer | | Start | Start | Start | Start | | Start | Start |
| Patient | Cancer | of | of | of | of | Days for | of | of |
| No. | Type | Culture | Culture | Culture | Culture | Culture | Culture | Culture |
| PT006 | Breast | 0.6% | 0.6% | 0.6% | 0.6% | 10 Days | 80.5 | N.T. |
| | Cancer | | | | | | | |
| ″ | Breast | 1.2% | 1.2% | 0.6% | 0.6% | 14 Days | 76.2 | 70.4 |
| | Cancer | | | | | | | |

As shown in Tables 13, 14, and 15, regardless of the plasma concentration in the culture medium, a CCR7⁺CD45RA⁺ cell population, a CD27⁺CD45RA⁺ cell population, and a CD28⁺CD45RA⁺ cell population were obtained by using the culture equipment immobilized with the anti-CD3 antibody and the CH-296 in the expansion of the lymphocytes. These cells have all phenotypes typical of naive T-like cells, and a high therapeutic effect against cancer may be expected when the lymphocytes after the expansion are given back to the body because of the accumulation of the cells in the lymph nodes, the rise of the cell viability in the body, and the differentiation to the cell with a high cytotoxicity against the cancer cells derived from the cancer patients. It was elucidated from this example that a cell population having a high therapeutic effect against cancer, wherein naive T-like cells had been expanded at a high rate, can be produced by using a combination of the anti-CD3 antibody and the CH-296 in the expansion of the lymphocytes using PBMCs of the cancer patient.

### Example 5

### Expansion of Lymphocytes from PBMCs of Cancer Patient (Culture with Use of Culture Flask)-4

### (1) Immobilization of OKT3 and CH-296

The OKT3 and the CH-296 were immobilized to the culture equipment used in the following experiment. That is, an ACD-A solution containing OKT3 (final concentration: 5 μg/mL) and CH-296 (final concentration: 25 μg/mL) was added to a 12-well cell culture plate (manufactured by Corning Inc.) in a volume of 0.45 mL/well each, and incubated at 37° C. for 5 hours in 5% CO₂. On this occasion, a group wherein only OKT3 was immobilized was set. The ACD-A solution containing the OKT3 or the OKT3 and the CH-296 was removed from the culture equipment by aspiration just before use, washed twice with DPBS and once with the RPMI 1640 medium, and then subjected to each experiment.

### (2) Expansion of Lymphocytes

The PBMC of 0.53×10⁶ cells derived from the cancer patient and separated in item (1) of Example 2 was suspended in 5.3 mL of 0.6% plasma containing KBM551 or 0.2% HSA/GT-T503, and added to the plate immobilized with the OKT3 or the OKT3 and the CH-296 as prepared in item (1) of Example 5. IL-2 was added thereto so as to have a final concentration of 200 U/mL and the mixture was incubated at 37° C. under 5% CO₂ (on day 0 of culture). On day 4 of culture, the cell solution in each well was suspended, and a part of the suspension was diluted 8.3-fold using 0.6% plasma-containing KBM551 or 0.2% HSA/GT-T503. Then, 7.8 mL of the diluted solution was transferred to a 25 cm²-cell culture flask (manufactured by Corning Inc.) to which nothing was immobilized, and added with IL-2 to each final concentration of 200 U/mL. The culture was continued, and on day 7, a plasma free KBM551 or a 0.2% HSA/GT-T503 was added to the culture solution in each group in an equivalent amount to dilute two-fold, and then added with IL-2 so as to have each final concentration of 200 U/mL. On day 10 of culture, the cell solution in each group was diluted two-fold using a plasma free KBM551 or a 0.2% HSA/GT-T503, and 15.6 mL each of the diluted solution was transferred to a fresh standing 25 cm²-cell culture flask to which nothing was immobilized. IL-2 was added thereto so as to have a final concentration of 200 U/mL in each group. On days 10 and 14 from the start of culture, the number of viable cells was counted by the trypan blue staining method and an expansion fold was calculated by comparing the number of counted cells with the number of cells at the start of culture. The results are shown in Table 16.

TABLE 16

| | | | | Expansion Fold (Fold) | |
|---|---|---|---|---|---|
| | | | | On Day 10 | On Day 14 |
| Cancer | Cancer | Initial | Basal | from Start | from Start |
| Patient No. | Type | Stimulation | Medium | of Culture | of Culture |
| PT003 | Breast | OKT3 | 0.2% HSA/ | ×200 | N.T. |
| | Cancer | | GT-T503 | | |
| ″ | Breast | OKT3 + CH-296 | 0.2% HSA/ | ×289 | N.T. |
| | Cancer | | GT-T503 | | |
| ″ | Breast | OKT3 | KBM551 | ×193 | N.T. |
| | Cancer | | | | |
| ″ | Breast | OKT3 + CH-296 | ″ | ×249 | N.T. |
| | Cancer | | | | |
| PT006 | Breast | OKT3 | KBM551 | ×202 | ×382 |
| | Cancer | | | | |
| ″ | Breast | OKT3 + CH-296 | ″ | ×386 | ×620 |
| | Cancer | | | | |
| PT008 | Laryngeal | OKT3 | KBM551 | ×119 | ×292 |
| | cancer | | | | |
| ″ | Laryngeal | OKT3 + CH-296 | ″ | ×229 | ×575 |
| | Cancer | | | | |
| PT012 | Rectal | OKT3 | KBM551 | ×281 | ×723 |
| | cancer | | | | |
| ″ | Rectal | OKT3 + CH-296 | ″ | ×532 | ×824 |
| | Cancer | | | | |

As shown in Table 16, regardless of the cancer type, a higher expansion rate of the lymphocytes was obtained with use of the culture equipment immobilized with the anti-CD3 antibody and the CH-296 in the expansion of the lymphocytes, compared to the group using the culture equipment immobilized with OKT3 alone, and an excellent effect was observed in the expansion of the lymphocytes derived from the cancer patients.

### (3) Analysis of CCR7⁺CD45RA⁺ Cells, CD62L⁺CD45RA⁺ Cells, and CCR7⁺CD62L⁺CD45RA⁺ Cells

With respect to the cells on days 10 and 14 of culture, prepared in item (2) of Example 5, analysis of CCR7⁺CD45RA⁺ cells, CD62L⁺CD45RA⁺ cells, and CCR7⁺CD62L⁺CD45RA⁺ cells was performed similarly to the method described in item (4) of Example 2. The results are shown in Tables 17, 18, and 19.

TABLE 17

| | | | | CCR7⁺CD45RA⁺ | |
|---|---|---|---|---|---|
| | | | | (%) | |
| | | | | On Day 10 | On Day 14 |
| Cancer | Cancer | Initial | Basal | from Start | from Start |
| Patient No. | Type | Stimulation | Medium | of Culture | of Culture |
| PT003 | Breast | OKT3 | 0.2% HSA/ | 13.3 | N.T. |
| | Cancer | | GT-T503 | | |
| ″ | Breast | OKT3 + CH-296 | 0.2% HSA/ | 28.1 | N.T. |
| | Cancer | | GT-T503 | | |
| ″ | Breast | OKT3 | KBM551 | 5.4 | N.T. |
| | Cancer | | | | |
| ″ | Breast | OKT3 + CH-296 | ″ | 11.0 | N.T. |
| | Cancer | | | | |
| PT006 | Breast | OKT3 | KBM551 | 24.9 | 36.9 |
| | Cancer | | | | |
| ″ | Breast | OKT3 + CH-296 | ″ | 41.8 | 69.0 |
| | Cancer | | | | |
| PT008 | Laryngeal | OKT3 | KBM551 | 4.6 | 4.2 |
| | cancer | | | | |
| ″ | Laryngeal | OKT3 + CH-296 | ″ | 7.3 | 17.2 |
| | Cancer | | | | |
| PT012 | rectal | OKT3 | KBM551 | 19.4 | 25.0 |
| | cancer | | | | |
| ″ | rectal | OKT3 + CH-296 | ″ | 36.0 | 40.1 |
| | Cancer | | | | |

TABLE 18

| | | | | CD62L⁺CD45RA⁺ | |
|---|---|---|---|---|---|
| | | | | (%) | |
| | | | | On Day 10 | On Day 14 |
| Cancer | Cancer | Initial | Basal | from Start | from Start |
| Patient No. | Type | Stimulation | Medium | of Culture | of Culture |
| PT003 | Breast | OKT3 | 0.2% HSA/ | 52.5 | N.T. |
| | Cancer | | GT-T503 | | |
| ″ | Breast | OKT3 + CH-296 | 0.2% HSA/ | 54.3 | N.T. |
| | Cancer | | GT-T503 | | |
| ″ | Breast | OKT3 | KBM551 | 50.9 | N.T. |
| | Cancer | | | | |
| ″ | Breast | OKT3 + CH-296 | ″ | 62.9 | N.T. |
| PT006 | Breast | OKT3 | KBM551 | 79.2 | 75.7 |
| | Cancer | | | | |
| ″ | Breast | OKT3 + CH-296 | ″ | 91.6 | 92.8 |
| | Cancer | | | | |
| PT008 | Laryngeal | OKT3 | KBM551 | 53.0 | N.T. |
| | cancer | | | | |
| ″ | Laryngeal | OKT3 + CH-296 | ″ | 59.7 | N.T. |
| | Cancer | | | | |
| PT012 | Rectal | OKT3 | KBM551 | 56.0 | 43.4 |
| | cancer | | | | |
| ″ | Rectal | OKT3 + CH-296 | ″ | 65.3 | 62.3 |
| | Cancer | | | | |

TABLE 19

| | | | | CCR7⁺CD62L⁺ | |
|---|---|---|---|---|---|
| | | | | CD45RA⁺ (%) | |
| | | | | On Day 10 | On Day 14 |
| Cancer | Cancer | Initial | Basal | from Start | from Start |
| Patient No. | Type | Stimulation | Medium | of Culture | of Culture |
| PT003 | Breast | OKT3 | 0.2% HSA/ | 13.1 | N.T. |
| | Cancer | | GT-T503 | | |
| ″ | Breast | OKT3 + CH-296 | 0.2% HSA/ | 27.7 | N.T. |
| | Cancer | | GT-T503 | | |
| ″ | Breast | OKT3 | KBM551 | 5.1 | N.T. |
| | Cancer | | | | |
| ″ | Breast | OKT3 + CH-296 | ″ | 10.9 | N.T. |
| | Cancer | | | | |
| PT006 | Breast | OKT3 | KBM551 | 24.6 | 36.7 |
| | Cancer | | | | |
| ″ | Breast | OKT3 + CH-296 | ″ | 41.7 | 68.9 |
| | Cancer | | | | |
| PT008 | Laryngeal | OKT3 | KBM551 | 4.2 | 3.9 |
| | cancer | | | | |
| ″ | Laryngeal | OKT3 + CH-296 | ″ | 7.2 | 17.0 |
| | Cancer | | | | |
| PT012 | Rectal | OKT3 | KBM551 | 17.1 | 23.4 |
| | cancer | | | | |
| ″ | Rectal | OKT3 + CH-296 | ″ | 30.9 | 38.9 |
| | Cancer | | | | |

As shown in Tables 17, 18, and 19, a CCR7⁺CD45RA⁺ cell population, a CD62L⁺CD45RA⁺ cell population, and a CCR7⁺CD62L⁺CD45RA⁺ cell population were obtained, regardless of the cancer type, by using the culture equipment immobilized with the anti-CD3 antibody and the CH-296 in the expansion of the lymphocytes, in comparison with the culture equipment immobilized with the anti-CD3 antibody alone. These cells have all phenotypes typical of naive T-like cells, and a high therapeutic effect against cancer may be expected when the lymphocytes after the expansion are given back to the body because of the accumulation of the cells in the lymph nodes, the rise of the cell viability in the body, and the differentiation to the cell with a high cytotoxicity against the cancer cells derived from the cancer patients. Moreover, the rate of the CCR7-positive cells is remarkably increased by the action of the anti-CD3 antibody and the CH-296 in the lymphocyte expansion in the example concerned, compared to the case where only OKT3 was made to act. The CCR7 is known as a receptor of CCL21 which is a chemokine in the lymph nodes, and CCR7 expression cells may be expected in antigen recognition in the lymph nodes and differentiation into cytotoxic lymphocytes. Accordingly, the lymphocytes prepared in accordance with the present invention are considered to be cells having a high ability of recognizing the cell derived from the cancer patients and attacking the cancer cell. In other words, it was elucidated from this example that a combination use of the anti-CD3 antibody and the CH-296 in the expansion of the lymphocytes using PBMCs of the cancer patients can produce a cell population with the CCR7-positive cells which had been proliferated at high efficiency, the cell population having a high therapeutic effect against cancers.

### Example 6

### Allogeneic Mixed Lymphocyte Reaction (MLR) Using Cultured Lymphocytes-1

### (1) Cryopreservation and Thawing of Lymphocytes Used

The cells on day 10 of culture, prepared by the expansion of the cells derived from cancer patient No. PT007 in item (3) of Example 2, were suspended in an RPMI 1640 medium, and a preservation solution of a 17:8 mixture of a cell preservation medium CP-1 (manufactured by Kyokuto Pharmaceutical Industrial Co., Ltd.) and 25% HSA was added in an equivalent amount, and then the resulting suspension was stored in liquid nitrogen. In the use of the preserved culture cells, they were promptly thawed at 37° C. in a water bath, washed with an RPMI 1640 medium containing 10 μg/mL DNase (manufactured by Calbiochem), stained by the trypan blue staining method to calculate the viable cell count, and then subjected to each experiment.

### (2) Separation and Storage of PBMC

Blood components in the blood cells were collected from a human healthy donor with informed consent, and the collected blood components were diluted two-fold with DPBS, overlaid on Ficoll-paque, and centrifuged at 700×g for 20 minutes. After the centrifugation, the PBMCs in the intermediate layer were collected with a pipette, and washed. The collected PBMCs derived from the donor were suspended in an RPMI 1640 medium so as to have a density of 5×10⁷ cells/mL, stored and thawed in liquid nitrogen in the same manner as in item (1) of Example 6, stained by the trypan blue staining method to calculate the viable cell count, and then subjected to each experiment.

### (3) Allogeneic MLR

An allogeneic MLR was carried out using the cells prepared in item (1) of Example 6 and item (2) of Example 6.

The cultured cells which had been thawed in item (1) of Example 6 were prepared with 5HRPMI so as to have a density of 2×10⁶ cells/mL and used as responder cells. On the other hand, PBMCs, prepared in item (2) of Example 6 and derived from an allogeneic donor (non-autologous donor: a healthy donor different from the patient in item (1) of Example 6), were irradiated with X-rays (0.88 C/kg) using an X-ray irradiation device (Type 260; manufactured by HITEX), washed with 5HRPMI, adjusted to 2×10⁶ cells/mL and used as stimulator cells.

The stimulator cells and the responder cells prepared so that each cell solution gave a cell ratio of 1:1 were added to a 24-well cell culture plate (manufactured by Corning Inc.) in a volume of 0.5 mL/well each. On this occasion, a group where only the responder cells were added was set. IL-2 was added to each well so as to have a final concentration of 500 U/mL, and culture was started at 37° C. in 5% CO₂ (on day 0 of culture). The autologous plasma concentration of the patient in the culture medium of the lymphocytes was set to the value as shown in Table 20.

On day 2 from the start of culture, 1 mL each of 5HRPMI was added to each well and IL-2 was added to a final concentration 500 U/mL (final cell culture volume: 2 mL/well).

On day 4 from the start of culture, the cells in each well were suspended, and the suspension was divided into two wells each having a half the volume, then 1 mL of 5HRPMI and IL-2 to a final concentration of 500 U/mL were added to all wells (final cell culture volume: 2 mL/well). The culture was continued until day 7 from the start of culture, and the viable cell count was counted by the trypan blue staining method. After that, the expansion rate of the cells were calculated in comparison with the cell count at the start of culture. The results are shown in Table 20.

TABLE 20

| Plasma | | |
|---|---|---|
| Concentration in | Ratio of Responder | |
| Culture Medium for | Cells and Stimulator | Expansion |
| Lymphocyte Culture | Cells | Fold (Fold) |
| 0.6% | 1:1 | x6.0 |
| ″ | 1:0 | x2.3 |
| 1.2% | 1:1 | x5.4 |
| ″ | 1:0 | x3.7 |

As shown in Table 20, it was elucidated that regardless of the plasma concentration in the lymphocyte culture, the lymphocytes obtained with use of the culture equipment immobilized with the anti-CD3 antibody and the CH-296 in the expansion of the lymphocytes increased non-self antigen-recognizing cells by the allogeneic MLR in the presence of the non-self antigens. This clarified that the cultured lymphocytes concerned have excellent effects in showing a high ability of recognizing the non-self antigen and exhibiting a proliferation ability specific to the non-self antigen.

### (4) Preparation of Phytohemaaglutinin (PHA) Blast Cells

The PBMCs derived from a non-autologous healthy donor, prepared in item (2) of Example 6, were adjusted with 5HRPMI to 1×10⁶ cells/mL, and seeded to a 6-well cell culture plate (manufactured by Corning Inc.) in a volume of 3 mL each. IL-2 was added thereto to a final concentration of 100 U/mL and phytohemaaglutinin (manufactured by Sigma, hereinafter referred to as PHA) was added thereto to a final concentration of 2 μg/mL, and the cells were cultured at 37° C. in 5% CO₂. On day 4 of culture, 12 mL of 5HRPMI was added to the cell culture and IL-2 was added thereto to a final concentration of 100 U/mL. Thereafter, the cell culture was dispensed into a 6-well cell culture plate so as to have a volume of 5 mL/well. The culture was continued until day 7 of culture, thereby preparing PHA blast (blastogenic) cells.

### (5) Determination of Non-Autologous Specific Cytotoxicity

Using the cells on day 7 of culture prepared in item (3) of Example 6, non-autologous specific cytotoxicity was assayed in the same manner as in item (5) of Example 2, except that the PHA blast cells which had been subjected to blastogenic cells in item (4) of Example 6 were used as the target cells which were incubated at 37° C. for 2 hours after addition of calcein-AM. These calcein-labeled target cells were mixed with a 30-fold amount of K562 cells, and the cytotoxicity was assayed using these target cells for determining the cytotoxicity. The K562 cells were used to exclude the non-specific cytotoxicity due to the NK cells contaminated in the responder cells prepared in item (1) of Example 6. The assay results are shown in Table 21.

TABLE 21

| Plasma Concentration in | | |
|---|---|---|
| Lymphocyte Culture Medium | E/T Ratio | Cytotoxicity (%) |
| 0.6% | 90 | 22.0 |
| ″ | 30 | 12.6 |
| ″ | 10 | 5.1 |
| ″ | 3 | 1.1 |
| 1.2% | 90 | 30.1 |
| ″ | 30 | 16.9 |
| ″ | 10 | 7.0 |
| ″ | 3 | 2.2 |

As shown in Table 21, it was elucidated that the lymphocytes obtained with use of the culture equipment immobilized with the anti-CD3 antibody and the CH-296 in the expansion of the lymphocytes can acquire a non-self antigen-specific cytotoxicity by the allogeneic MLR with the non-autologous cells, regardless of the plasma concentration in the lymphocyte culture, thereby demonstrating a strong immunity.

### Example 7

### Allogeneic Mixed Lymphocyte Reaction (MLR) Using Cultured Lymphocytes-2

### (1) Cryopreservation and Thawing of Lymphocytes Used

The same method as in item (1) of Example 6 was carried out using the cells on days 10 and 14 from the start of culture, prepared by the expansion of the cells derived from the cancer patient No. PT012 in item (3) of Example 2. Here, a group where only OKT3 was immobilized to the culture equipment in the expansion of the lymphocytes was also set.

### (2) Allogenic MLR

An allogeneic MLR was carried out in a similar manner to item (3) of Example 6, using the cancer patient-derived autologous lymphocytes prepared in item (1) of Example 7 and the human healthy donor-derived non-autologous PBMCs prepared similarly to item (2) of Example 6. The results are shown in Table 22.

TABLE 22

| Days of | | | |
|---|---|---|---|
| Lymphocyte | Initial | Ratio of Responder | Expansion |
| Expansion | Stimulation | cell:Stimulator cell | Rate (Fold) |
| 10 Days | OKT3 | 1:1 | x5.3 |
| ″ | OKT3 + | ″ | x6.3 |
| | CH-296 | | |
| 14 Days | OKT3 | ″ | x3.7 |
| ″ | OKT3 + | ″ | x4.8 |
| | CH-296 | | |
| 10 Days | OKT3 | 1:0 | x3.0 |
| ″ | OKT3 + | ″ | x2.8 |
| | CH-296 | | |
| 14 Days | OKT3 | ″ | x1.1 |
| ″ | OKT3 + | ″ | x0.9 |
| | CH-296 | | |

As shown in Table 22, when compared to the group with use of the culture equipment immobilized with the OKT3 alone, it was elucidated that the lymphocytes obtained with use of the culture equipment immobilized with the anti-CD3 antibody and the CH-296 in the expansion of the lymphocytes can increase the non-self antigen-recognizing cells at a higher expansion fold by the allogeneic MLR in the presence of non-autologous cells. In addition, it was shown that even a short term lymphocyte expansion had a sufficient effect.

### (3) Preparation of PHA Blast Cells

Using the PBMCs derived from an allogeneic donor, prepared in item (2) of Example 6, the same method as in item (4) of Example 6 was carried out.

### (4) Determination of Non-Autologous Specific Cytotoxicity

Using the cells on day 7 prepared in item (2) of Example 7, the same method as in item (5) of Example 6 was carried out to assay the non-autologous specific cytotoxicity. The assay results are shown in Table 23.

TABLE 23

| Days of Lymphocyte | | | Cytotoxicity |
|---|---|---|---|
| Expansion | Initial Stimulation | E/T Ratio | (%) |
| 10 Days | OKT3 | 90 | 15.2 |
| ″ | OKT3 + CH-296 | ″ | 19.1 |
| 14 Days | OKT3 | ″ | 27.6 |
| ″ | OKT3 + CH-296 | ″ | 29.9 |

As shown in Table 23, when compared to a group with use of the culture equipment immobilized with the OKT3 alone, it was elucidated that the lymphocytes obtained with use of the culture equipment immobilized with the OKT3 and the CH-296 in the expansion of the lymphocytes showed a higher non-self antigen-specific cytotoxicity by the allogeneic MLR, regardless of the days for the lymphocyte expansion.

### Example 8

### Examination of Resistance to Various Anticancer Agents Using Cultured Lymphocytes-1

### (1) Lymphocytes Used

The cells on day 10 from the start of culture, derived from the cancer patient No. PT009 and prepared in item (3) of Example 2, were subjected to cryopreservation, thawing and washing in a similar manner to item (1) of Example 6, and suspended in 5HRPMI containing IL-2 (final concentration of 222 U/mL) (hereinafter referred to as IL-2/5HRPMI), then passed through a 40 μm cell strainer. After that, the suspension was stained by the trypan blue staining method to calculate the viable cell count, and then subjected to each experiment.

### (2) Examination of Resistance to Anticancer Agents

The IL-2/5HRPMI was added to a 96-well cell culture plate (manufactured by Corning Inc.) in 130 μL/well each and serially diluted test drugs were added thereto in 20 μL/well each, using carboplatin (drug name: Paraplatin Injection, manufactured by Bristol-Myers), fluorouracil injection (drug name: 5-FU Injection 250 Kyowa, manufactured by Kyowa Hakko Kogyo Co., Ltd.), cisplatin (drug name: Cisplatin Injection “Nichi-Iko”, manufactured by Nichi-Iko Pharmaceutical Co., Ltd.), vincristine sulfate (drug name: Oncovine Injection, manufactured by Nippon Kayaku Co., Ltd.), doxorubicin hydrochloride (drug name: Adriacin Injection 10, manufactured by Kyowa Hakko Kogyo Co., Ltd.), and dexamethasone sodium phosphate (drug name: Decadoron Injection, manufactured by Banyu Pharmaceutical Co., Ltd.). The cells prepared in item (1) of Example 8 were adjusted with the IL-2/5HRPMI to have a density of 4×10⁶ cells/mL and added to the test cell addition group in 50 μL/well each (2×10⁴ cells/well). On the other hand, the IL-2/5HRPMI was added to the control group in 50 μL/well each.

These culture plates were incubated at 37° C. for 20 hours in the presence of 5% CO₂. After the incubation, WST-1 (Premix WST-1 Cell Proliferation Assay System, manufactured by Takara Bio Inc.) was added in 20 μL/well each to each well of the plate, and incubated in the presence of 5% CO₂ at 37° C. for 4 hours. The plates after the incubation were centrifuged at 500×g at room temperature for 5 minutes. After the centrifugation, 100 μL of the supernatant was collected from each well, and the absorbance was measured at an absorption wavelength of 450 nm and a control wavelength of 630 nm [hereinafter referred to as the absorbance (450 nm-630 nm)] using a microplate reader (manufactured by Bio-Rad Laboratories, Inc., Model Number 680XR), and then the specific absorbance (450 nm-630 nm) was calculated according to the following formula.

Specific absorbance (450 nm-630 nm)=Absorbance of Test Cell Addition Group (450 nm-630 nm)−Absorbance of Control Group (450 nm-630 nm) Formula 2:

Based on the specific absorbance (450 nm-630 nm) at each concentration of anticancer agents, the concentration of the anticancer agent causing 50% inhibition of the cell growth was calculated as the growth inhibitory concentration of 50% inhibition of growth (hereinafter referred to as GI₅₀), and a resistance examination to various anticancer agents was performed. The results are shown in Table 24.

TABLE 24

| Anticancer Agent | GI₅₀ (μg/mL) |
|---|---|
| Carboplatin | 215.5 |
| Fluorouracil | 639.3 |
| Cisplatin | 3.7 |
| Vincristine Sulfate | 0.2 |
| Doxorubicin Hydrochloride | 0.5 |
| Dexamethasone Sodium Phosphate | 38.9 |

As shown in Table 24, the GI₅₀ value to each anticancer agent was higher than the remaining blood concentration at post-administration which had been generally reported for each anticancer agent. Therefore, the lymphocytes obtained by using the culture equipment immobilized with the anti-CD3 antibody and the CH-296 in lymphocyte expansion showed a proliferative property even in the presence of various anticancer agents. It was clarified from this example that a cell group resistant to anticancer agents can be obtained by using the anti-CD3 antibody and the CH-296 in lymphocyte expansion of cancer patients' PBMC, and an adoptive immunotherapy using the cell population is effective in combinatorial cancer therapy with use of anticancer agents.

### Example 9

### Examination of Resistance to Various Anticancer Agents Using Cultured Lymphocytes-2

### (1) Expansion of Lymphocytes Used

In a similar manner to item (3) of Example 2, the expansion of the lymphocytes derived from a human healthy donor was carried out, and the viable cell count was counted by the trypan blue staining method in a similar manner to item (1) of Example 2, and then the cells were subjected to each experiment. Here, a group where only OKT3 was immobilized to the culture equipment in the expansion of the lymphocytes was also set.

### (2) Examination of Resistance to Anticancer Agents

In a similar manner to item (2) of Example 8, a resistance examination to anticancer agents was carried out, except that carboplatin, fluorouracil injection, vincristine sulfate, doxorubicin hydrochloride, dexamethasone sodium phosphate, and paclitaxel injection (drug name: Taxol, manufactured by Bristol-Myers) were used as the test drugs, and they were serially diluted and added respectively in 20 μL/well each. However, in the case of paclitaxel, a human AB type serum was used as a solvent for dilution, and for other drugs, 5HRPMI was used for dilution. The results are shown in Table 25.

TABLE 25

GI₅₀ (μg/mL)

| | Initial | Initial |
|---|---|---|
| | Stimulation | Stimulation |
| Anticancer Agent | OKT3 | OKT3 + CH-296 |
| Carboplatin | 116.16 | 161.31 |
| Fluorouracil | 2293.08 | 3273.11 |
| Vincristine Sulfate | 4.42 | 24.34 |
| Doxorubicin Hydrochloride | 0.25 | 0.41 |
| Dexamethasone Sodium | 86.33 | 118.97 |
| Phosphate | | |
| Paclitaxel | 34.08 | 39.73 |

As shown in Table 25, the lymphocytes obtained by using the culture equipment immobilized with the anti-CD3 antibody and the CH-296 in lymphocyte expansion showed a proliferative property even in the presence of various anticancer agents. In addition, the drug resistance was higher than that of the group where the culture equipment immobilized with OKT3 alone was used. It was clarified from this example that a cell group with much more resistant to anticancer agents can be obtained by using the anti-CD3 antibody and the CH-296 in lymphocyte expansion with use of PBMCs, and an adoptive immunotherapy using the cell population was effective in combinatorial cancer therapy with anticancer agents.

### Example 10

### Studies on Effects of Transferred Naive T-Like Cells after Administration of Anticancer Agent Using Mouse Syngeneic Tumor Model-1

### (1) Expansion of Mouse T Cell Populations

In a similar manner to item (5) of Example 1, expansion of mouse T cells was carried out, however, the human CH-296 was not used, and the cells on day 6 of culture were collected. The cells were subjected to each experiment.

### (2) Separation of Naive T-Like Cells and Effector T-Like Cells in Expanded Mouse T Cell Population

The cells obtained in item (1) of Example 10 were collected to take a required amount, and centrifuged at 500×g for 5 minutes at room temperature to remove the supernatant. Thereafter, the cells were suspended in DPBS containing 0.5% BSA and 2 mM disodium ethylenediaminetetraacetate (hereinafter referred to as 0.5% BSA/DPBS) to a density of 1.11×10⁸ cells/mL. CD62L (L-secretin) microbeads (mouse) (manufactured by MACS) were added to the cell solution in 10 μL per cell count of 1×10⁷ cells, and incubated for 15 minutes while stirring sometimes in a dark place at 4° C. Then, to the cell solution was added 1 mL of 0.5% BSA/DPBS per cell count of 1×10⁷ cells, and the mixture was centrifuged at 500×g for 5 minutes at room temperature to remove the supernatant. After that, 0.5 mL of 0.5% BSA/DPBS per cell count of 1×10⁸ cells was added thereto, suspended sufficiently, and allowed to stand on ice to afford a CD62L microbeads target cell solution. An LS column (manufactured by MACS, hereinafter referred to as a separation column) was provided to a VarioMACS (trademark) separator (manufactured by MACS, hereinafter referred to as a separation apparatus) and rinsed with 3 mL of 0.5% BSA/DPBS. The CD62L microbeads-labeled cell solution was added to the column for elution and further rinsed with 9 mL of 0.5% BSA/DPBS to collect an eluate, thereby obtaining CD62L⁻ cells as effector T-like cells. The column was removed from the separation apparatus, and CD62L⁺ cells were collected as naive T-like cells by extrusion with a plunger attached to the separation apparatus after addition of 5 mL of a buffer.

(3) Anticancer Agent Administration and T Cell Population Transfer in Syngeneic Tumor Model of C57BL/6-hB16F10

Hair removal of about 9 cm² in the right inguina of 7-week old female C57BL/6 mice was effected under anesthesia, and 0.1 mL of hB16F10 suspended in an RPMI 1640 medium to a density of 4×10⁶ cells/mL was subcutaneously administered to the mice. Thereafter, group settings were carried out as follows. Group A was set to an untreated group, group B was set to an MMC alone administration group, group C was set to a combinatorial MMC and naive T-like cell administration group, and group D was set to a combinatorial MMC and effector T-like cell administration group. On days 3 and 4 after tumor inoculation, 0.2 mL of a saline (manufactured by Otsuka Pharmaceutical Co., Ltd.) was intraperitoneally administered to group A and the MMC at a dose of 2 mg/kg was intraperitoneally administered to other groups. On day 6 after the tumor inoculation, individual mouse T cells prepared in item (2) of Example 10 were prepared with an RPMI 1640 medium to a density of 5×10⁸ cells/mL. The naive T-like cells and the effector T-like cells in 0.2 mL each were respectively administered to each individual of group C and group D via the tail veins. In addition, the RPMI 1640 medium in 0.2 mL each was administered via the tail veins to each individual of group A and group B.

(4) Evaluation of Transferred T Cell Population after Administration of Anticancer Agent in Syngeneic Tumor Model of C57BL/6-hB16F10

Evaluation of the lymphocyte count was effected by measuring the leukocyte count and the T cell count contained in the blood sample collected from the mouse tail vein. A volume of 22 μL of the blood was collected into a 0.5 mL tube containing 3 μL of heparin sodium (manufactured by Mitsubishi Wellpharma Inc.) per one mouse. An aliquote of 15 μL was taken from the blood sample, added to a mixed solution of 14 μL of Flow-Count (manufactured by Beckman Coulter Inc.) and 0.5 μL of hamster anti-mouse CD3e FITC (manufactured by eBioscience Inc.), and the mixture was treated for 15 minutes. Thereafter, the erythrocytes were hemolysed with a low isotonic solution, and subjected to flow cytometry to calculate the rate of T cells and the recovery rate of T cells, with the CD3e⁺ cells being as the T cells. The assay was performed immediately before administering the cells and on day 4 after administering the cells. In addition, the rate of the T cells shows the rate when the number of the T cells of group A (untreated group) was defined as 100%. Moreover, the recovery rate of the T cells was determined to be a ratio of the number of the T cells on day 4 after the cell administration relative to the number of the T cells immediately before the cell administration. The results are shown in Table 26 and Table 27.

TABLE 26

| | Rate of T Cells | |
|---|---|---|
| | Immediately | On Day 4 after |
| | before Cell | Cell |
| | Administration | Administration |
| Group A Untreated Group | 100 | 100 |
| Group B MMC | 84.1 | 118.2 |
| Group C MMC + Naive T-Like Cells | 80.8 | 143.7 |
| Group D MMC + Effector T-Like | 84.1 | 138.5 |
| Cells | | |

TABLE 27

| | Recovery Rate |
|---|---|
| | of T Cells |
| Group A Untreated Group | 1.0 |
| Group B MMC | 1.4 |
| Group C MMC + Naive T-Like Cells | 1.8 |
| Group D MMC + Effector T-Like | 1.6 |
| Cells | |

As shown in Tables 26 and 27, a combination use of the MMC administration and the naive T-like cell administration in a syngeneic tumor model showed a higher rate of T cells and a higher recovery rate of T cells, compared to those in the case of a combinatorial administration of the MMC and the effector T-like cells. This demonstrates that a survival rate of the naive T-like cells in the living body is high. It was shown from this example that a combination of the anticancer agent administration and the expanded naive T-like cell administration can recover the lymphocyte count reduction caused by the administration of anticancer agents early. In addition, it was confirmed that an adoptive immunotherapy using a cell population of naive T-like cells which had been grown at high efficiency is extremely effective in cancer therapy with a combination use of anticancer agents.

(5) Evaluation of Antitumor Activity after Administration of Anticancer Agent and T Cells in Syngeneic Tumor Model of C57BL/6-hB16F10

In order to assay the antitumor activity in the evaluation system performed in item (3) of Example 10, a tumor size in each individual on day 14 after the tumor inoculation was determined with an electronic caliper. The results are shown in Table 28 (The tumor size is shown as a product of the major diameter and the minor diameter of the tumor).

TABLE 28

| | | Standard |
|---|---|---|
| | Tumor Size (mm²) | Error |
| Group A Untreated Group | 98.6 | 8.5 |
| Group B MMC | 82.5 | 13.0 |
| Group C MMC + Naive T-Like Cells | 55.6 | 5.8 |
| Group D MMC + Effector T-Like | 78.4 | 8.0 |
| Cells | | |

As shown in Table 28, a combination use of the MMC administration and the naive T-like cell administration in a syngeneic tumor model resulted in a smaller tumor size and a higher antitumor activity, compared to the case where the MMC and the effector T-like cells were administered in combination. From this example, combination administration of the anticancer agent and the expanded naive T-like cells showed an inhibitory activity against the tumor growth due to the effect of combination use with the anticancer agent. In addition, it was confirmed that an adoptive immunotherapy using a cell population of naive T-like cells which had been grown at high efficiency is extremely effective in cancer therapy with a combination use of anticancer agents.

### Example 11

### Studies on Effects of Transferred T-Like Cells after Administration of Anticancer Agent Using Mouse Syngeneic Tumor Model-2

### (1) Expansion of Mouse T Cell Population

In a similar manner to item (5) of Example 1, expansion of mouse T cell population was carried out.

### (2) Separation of Naive T-Like Cells and Effector T-Like Cells in Expanded Mouse T Cell Population

Using the mouse T cells which had been expanded in item (1) of Example 11, separation of the naive T-like cells and the effector T-like cells was performed similarly to item (2) of Example 10.

(3) Anticancer Agent Administration and T-Cell Population Transfer in Syngeneic Tumor Model of C57B/6-hB16F10

In this study, cyclophosamide (drug name; Endoxane, manufacture by Shionogi & Co., Ltd., hereinafter referred to as CPA) was used as an anticancer agent. In a similar manner to item (3) of Example 10, the hB16F10 was subcutaneously administered to mice. Thereafter, group settings were carried out as follows. Group A was set to an untreated group, group B was set to a CPA alone administration group, group C was set to a combinatorial CPA and naive T-like cell administration group, and group D was set to a combinatorial CPA and effector T-like cell administration group. On day 4 after the tumor inoculation, 0.2 mL of a saline was intraperitoneally administered to group A, and the CPA at a dose of 100 mg/kg was intraperitoneally administered to other groups. On the next day, individual mouse T cells prepared in item (2) of Example 11 was prepared with an RPMI 1640 medium to a density of 3.75×10⁸ cells/mL, and the naive T-like cells and the effector T-like cells in 0.2 mL each were administered via the tail veins to each individual of group C and group D, respectively. In addition, the RPMI 1640 medium was administered to each individual of group A and group B in 0.2 mL each via the tail veins.

(4) Evaluation of Transferred T Cell Population after Administration of Anticancer Agent in Syngeneic Tumor Model of C57BL/6-hB16F10

Evaluation of the lymphocyte count was conducted similarly to item (4) of Example 10. However, the measurement was done on day 6 after the cell administration. The results are shown in Table 29.

TABLE 29

| | Rate of T |
|---|---|
| | cells (%) |
| Group A | 100 |
| Group B CPA | 52.2 |
| Group C CPA + Naive T-Like Cells | 68.6 |
| Group D CPA + Effector T-Like | 43.5 |
| Cells | |

As shown in Table 29, a combination of the CPA administration and the naive T-like cell administration in a syngeneic tumor model showed a high rate of T-cells after the cell administration, compared to that in the case where the CPA alone or a combination of the CPA and the effector T-like cells was administered. This demonstrates that a survival rate of the naive T-like cells in the living body is high. It was shown from this example that a combination of the anticancer agent administration and the expanded naive T-like cell administration can recover the lymphocyte count reduction caused by the administration of anticancer agents early. In addition, it was confirmed that an adoptive immunotherapy using a cell population of naive T-like cells which had been grown at high efficiency is extremely effective in cancer therapy in combination with anticancer agents.

(5) Evaluation of Antitumor Activity after Administration of Anticancer Agent and T Cells in Syngeneic Tumor Model of C57BL/6-hB16F10

The antitumor activity in the evaluation system performed in item (3) of Example 11 was evaluated in a similar manner to the method described in item (5) of Example 10. However, the tumor size was determined on days 11 and 13 after the tumor inoculation. The results are shown in Table 30.

TABLE 30

Tumor Size (mm²)

| | On day 11 | On day 13 |
|---|---|---|
| Group A | 46.6 | 78.6 |
| Group B CPA | 32.6 | 47.3 |
| Group C CPA + Naive T-Like Cells | 26.4 | 40.4 |
| Group D CPA + Effector T-Like | 33.9 | 44.3 |
| Cells | | |

As shown in Table 30, a combination use of the CPA administration and the naive T-like cell administration in a syngeneic tumor model showed a small tumor size on both measurement days, indicating a result of higher tumor activity, compared to the case where the CPA alone or a combination of the CPA and the effector T-like cells was administered. From this example, combination for use in administration of the anticancer agent and the expanded naive T-like cells showed a tumor growth inhibitory activity due to the combination use effect with the anticancer agent. In addition, it was confirmed that an adoptive immunotherapy using a cell population of naive T-like cells which had been proliferated at high efficiency is extremely effective in cancer therapy with a combination use of anticancer agents.

### INDUSTRIAL APPLICABILITY

According to the present invention, a cancer therapy and a therapeutic agent for cancer, having an activated cell-mediated immunity against cancer and a high therapeutic effect, are provided. Moreover, the present treatment method and the therapeutic agent can also reduce the risks of infectious diseases because reduction in the immunity due to decrease in the number of the lymphocytes can be avoided.

### Sequence Listing Free Text

SEQ ID NO:1; Partial region of fibronectin named III-8.

SEQ ID NO:2; Partial region of fibronectin named III-9.

SEQ ID NO:3; Partial region of fibronectin named III-10.

SEQ ID NO:4; Partial region of fibronectin named III-11.

SEQ ID NO:5; Partial region of fibronectin named III-12.

SEQ ID NO:6; Partial region of fibronectin named III-13.

SEQ ID NO:7; Partial region of fibronectin named III-14.

SEQ ID NO:8; Partial region of fibronectin named CS-1.

SEQ ID NO:9; Fibronectin fragment named C-274.

SEQ ID NO:10; Fibronectin fragment named H-271.

SEQ ID NO:11; Fibronectin fragment named H-296.

SEQ ID NO:12; Fibronectin fragment named CH-271.

SEQ ID NO:13; Fibronectin fragment named CH-296.

SEQ ID NO:14; Fibronectin fragment named C-CS1.

SEQ ID NO:15; Fibronectin fragment named CH-296Na.

SEQ ID NO:16; Fibronectin fragment named CHV-89.

SEQ ID NO:17; Fibronectin fragment named CHV-90.

SEQ ID NO:18; Fibronectin fragment named CHV-92.

SEQ ID NO:19; Fibronectin fragment named CHV-179.

SEQ ID NO:20; Fibronectin fragment named CHV-181.

SEQ ID NO:21; Fibronectin fragment named H-275-Cys.

SEQ ID NO:22; Fibronectin fragment named H296-H296.

SEQ ID NO:23; Fibronectin fragment named H105-H105.

1. A method of treating cancer, comprising the following steps (A) and (B):
(A) applying a treatment which induces the reduction in lymphocytes to a patient; and
(B) subsequent to the step (A), administering lymphocytes to the patient promptly.

2. The treatment method according to claim 1, wherein the treatment which induces the reduction in lymphocytes to a patient is administration of an anticancer agent and/or radiation.

3. The treatment method according to claim 2, wherein the anticancer agent is at least one anticancer agent selected from a group consisting of anticancer agents classified into a metabolic antagonist, an antibiotic (an antitumor antibiotic), a microtubule inhibitor, a topoisomerase inhibitor, a platinum preparation, an alkylating agent or a corticosteroid.

4. The treatment method according to claim 1, wherein the step (B) is carried out one hour to 10 days after the step (A).

5. The treatment method according to claim 1, wherein the lymphocytes to be administered are a culture.

6. The treatment method according to claim 5, wherein the lymphocytes to be administered are a lymphocyte culture obtained by culturing lymphocytes collected from a patient.

7. The treatment method according to claim 5, wherein the lymphocytes to be administered are a lymphocyte culture obtained by culturing lymphocytes in the presence of an anti-CD3 antibody.

8. The treatment method according to claim 5, wherein the lymphocytes to be administered are a lymphocyte culture obtained by culturing lymphocytes in the presence of fibronectin, a fibronectin fragment, or a mixture thereof.

9. The treatment method according to claim 1, further comprising the step of administering a cancer vaccine and/or a lymphocyte stimulating factor during or after the step (B).

10. A lymphocyte-containing therapeutic agent for cancer, which can be administered promptly to a patient to whom a treatment inducing the reduction in lymphocytes has been applied, subsequent to the treatment.

11. The therapeutic agent according to claim 10, wherein the treatment inducing the reduction in lymphocytes is administration of an anticancer agent and/or radiation.

12. The therapeutic agent according to claim 10, which can be administered to a patient to whom a treatment inducing the reduction in lymphocytes has been applied, one hour or 10 days after the treatment.

13. The therapeutic agent according to claim 10, wherein the lymphocytes are a culture.

14. The therapeutic agent according to claim 13, wherein the lymphocytes are a lymphocyte culture obtained by culturing lymphocytes collected from a patient.

15. The therapeutic agent according to claim 13, wherein the lymphocytes are a lymphocyte culture obtained by culturing lymphocytes in the presence of an anti-CD3 antibody.

16. The therapeutic agent according to claim 13, wherein the lymphocytes are a lymphocyte culture obtained by culturing lymphocytes in the presence of fibronectin, a fibronectin fragment, or a mixture thereof.

17. A cancer treatment kit, containing separately an anticancer agent which causes the reduction in lymphocytes and the therapeutic agent according to claim 10.

18. The cancer treatment kit according to claim 17, wherein the anticancer agent is at least one anticancer agent selected from a group consisting of anticancer agents classified into a metabolic antagonist, an antibiotic (an antitumor antibiotic), a microtubule inhibitor, a topoisomerase inhibitor, a platinum preparation, an alkylating agent or a corticosteroid.

19. A cancer treatment kit, containing separately the therapeutic agent according to claim 10, and a cancer vaccine and/or a lymphocyte stimulating factor.

20. A cancer treatment kit, containing separately the cancer treatment kit according to claim 17, and a cancer vaccine and/or a lymphocyte stimulating factor.

21. A method of producing the therapeutic agent according to claim 10 using lymphocytes.

22. A method of producing a cancer treatment kit according to claim 17, using an anticancer agent which causes the reduction in lymphocytes.

23. A method of producing a cancer treatment kit according to claim 19, using lymphocytes, and a cancer vaccine and/or a lymphocyte stimulating factor.

24. A method of producing a cancer treatment kit according to claim 20, using an anticancer agent which causes the reduction in lymphocytes, lymphocytes, and a cancer vaccine and/or a lymphocyte stimulating factor.
